(19) United States
(12) Patent Application Publication
(10) Pub. No.: US 2025/0114311 A1
(43) Pub. Date: Apr. 10, 2025

(54) IONIZABLE CATIONIC COMPOUNDS FOR MESSENGER RNA DELIVERY

(71) Applicant: SEQIRUS INC., Holly Springs, NC (US)

(72) Inventor: Eric L. DANE, Holly Springs, NC (US)

(21) Appl. No.: 18/695,917

(22) PCT Filed: Sep. 28, 2022
(86) PCT No.: PCT/IB2022/059217

§ 371 Date: Mar. 27, 2024

Related U.S. Application Data
(60) Provisional application No. 63/249,128, filed on Sep. 28, 2021.

(51) Int. Cl. A61K 9/51; A61K 39/00; A61K 39/145; A61K 39/215; C12N 15/88

(52) U.S. Cl. CPC A61K 9/5123; A61K 9/5146; A61K 39/0011; A61K 39/145; A61K 39/215; C12N 15/88; A61K 2039/53; A61K 2039/6018

(57) ABSTRACT

The use of certain ionizable cationic lipid compounds in forming lipid nanoparticles is described. The lipid nanoparticles may encapsulate a therapeutic, such as a nucleic acid, and may be used in the delivery of the therapeutic and in methods of treating certain conditions, or for inducing an immune response.

### FIELD

The present disclosure relates to cationic and/or ionizable lipid compounds that can be used, in combination with other lipid molecules, to form lipid nanoparticles for delivery of a messenger RNA to a subject.

### BACKGROUND

Nucleic acid-based therapies have shown substantial promise in a range of therapeutic applications. The delivery of polynucleotides such as messenger RNA (mRNA), self-amplifying mRNA (saRNA), small interfering RNA (siRNA), antisense oligonucleotides, plasmids, DNA and the like does, however, present a number of challenges. Free nucleic acids, such as RNAs, are subject to rapid enzymatic degradation and so generally do not persist systemically. Additionally, due to their negative charge the nucleic acids may not be able to effectively cross the cellular barriers to enter the necessary intracellular compartment, for example, for translation or to otherwise achieve their effect.

Lipid particles such as lipid nanoparticles (LNPs) have therefore been used to formulate nucleic acids so as to protect them from degradation and improve cellular uptake and intracellular delivery. LNPs are commonly formed from ionizable cationic lipids and other lipid components such as neutral lipids, sterols such as cholesterol and PEGylated lipids. Ionizable cationic lipids are amphiphilic molecules having a lipophilic region containing one or more hydrocarbon groups and a hydrophilic region containing at least one positively charged or ionizable polar head group. Such cationic lipids are ionized at an appropriate pH and can then form a positively charged complex with nucleic acids, making it easier for the nucleic acids to pass through the plasma membrane of the cell and enter the cytoplasm.

The first siRNA therapeutic to be approved, Onpattro (patisiran), entered the market just a few years ago for treatment of hereditary amyloidogenic transthyretin (TTR) amyloidosis. Patisiran's therapeutic effect relies on siRNA-mediated TTR gene silencing, preventing mutant protein production to at least prevent disease progression. The efficient delivery of the siRNA depends upon the LNP technology.

Even more recently, nucleic acid vaccines have emerged as a promising approach to the treatment and prevention of various diseases, including against the severe acute respiratory syndrome coronavirus 2 (SARS-CoV-2) responsible for causing the on-going worldwide pandemic of the severely infectious coronavirus disease 2019 (COVID-19). Messenger RNA vaccines rely on the delivery of conventional mRNA or self-amplifying messenger RNA (saRNA) into the cytoplasm of host cells, where it is eventually transcribed and translated into antigenic proteins to trigger the production of an immune response. The large size and negative charge of messenger RNA prevents cellular uptake and so LNPs are again necessary for appropriate delivery.

Different ionizable cationic lipids may present not only different physicochemical profiles, including their acid dissociation constant (pKa) value, thereby affecting their ability to complex with the nucleic acid, but also different toxicity profiles in vivo and so it will be apparent to the skilled person that there is an on-going need for improved ionizable cationic lipid compounds which are suitable to form lipid particles, such as LNPs, for delivery of messenger RNA.

### BRIEF SUMMARY

Embodiments of the present invention provide for ionizable cationic lipid compounds, and pharmaceutically acceptable salts, and prodrugs thereof, which can form lipid particles, for example LNPs, in the presence of additional lipids including one or more of neutral lipids, charged lipids, structural lipids, PEGylated lipids and analogs thereof, and which can be used for the delivery of messenger RNA, including conventional messenger RNA and self-amplifying messenger RNA (saRNA). The lipid particles comprising the ionizable cationic lipid compounds and associated messenger RNA may be used in the treatment of a range of diseases, disorders and conditions.

Accordingly, in one embodiment, the present disclosure provides for a lipid nanoparticle associated with a messenger RNA, the lipid nanoparticle comprising one or more ionizable cationic lipid compounds selected from the group consisting of:

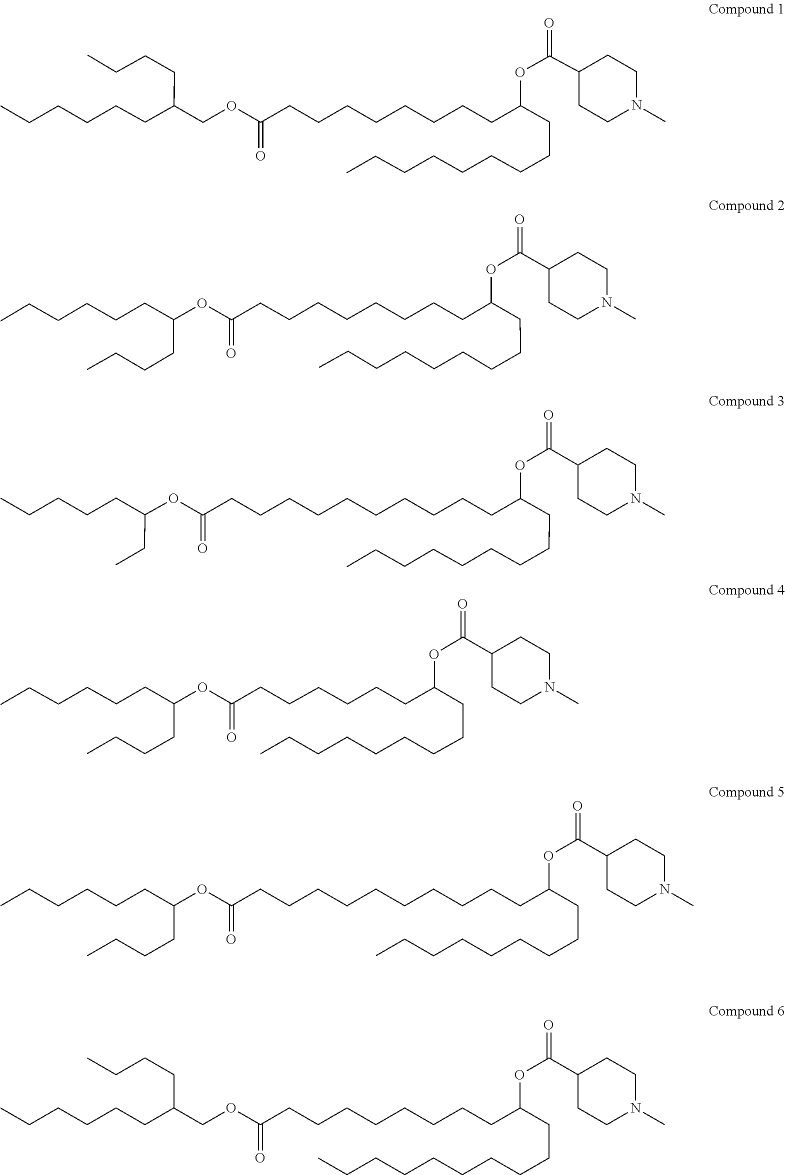

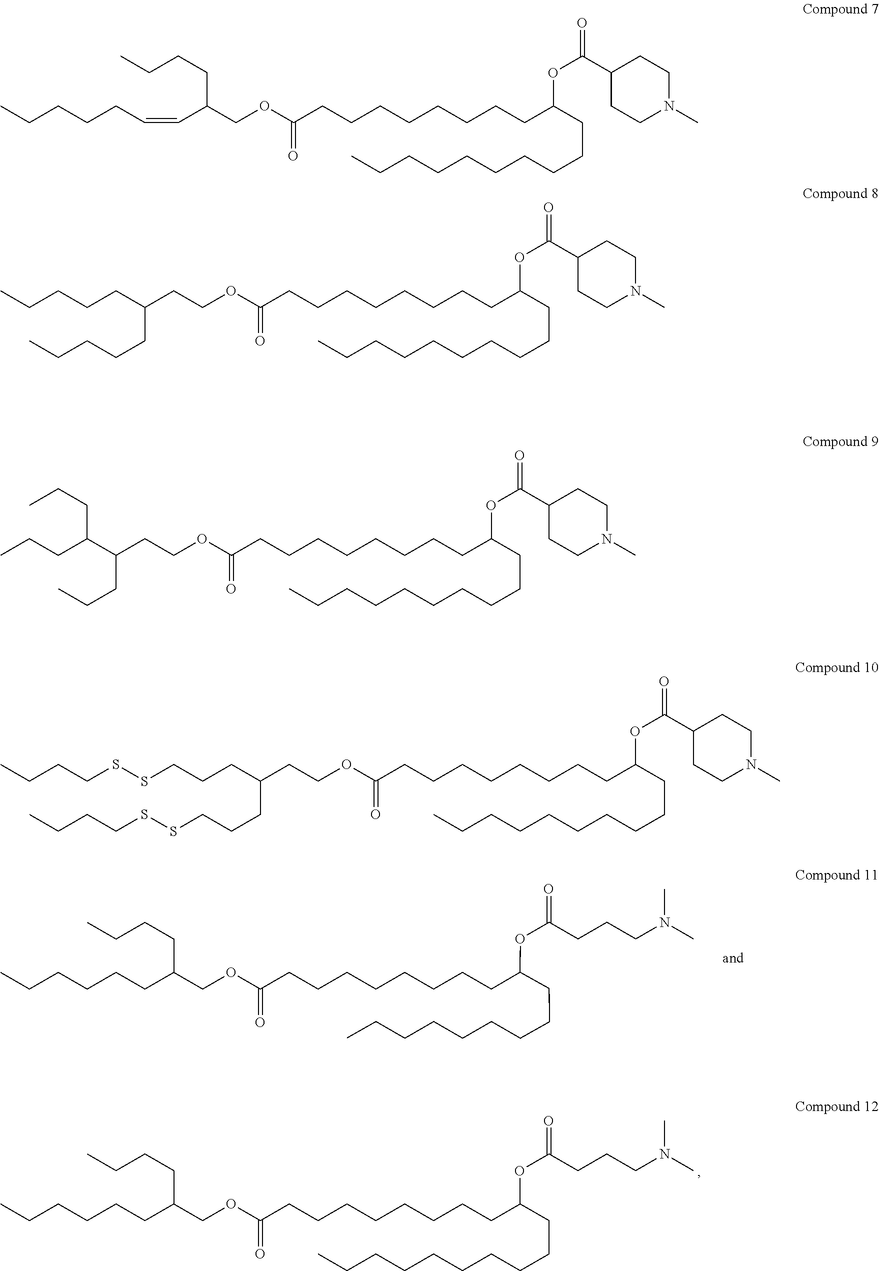

or pharmaceutically acceptable salt or prodrug thereof.

In a further embodiment, the present disclosure provides for a lipid particle composition, such as an LNP composition, comprising a plurality of such lipid particles and at least one pharmaceutically acceptable carrier, diluent or excipient.

In a further embodiment, a method of delivering a messenger RNA, within the foregoing lipid particle and/or lipid particle composition, to a cell is provided.

In further embodiments, a method of producing a polypeptide of interest in a cell is provided.

In still further embodiments, a method of treating a disease, disorder or condition in a subject is provided by administering the foregoing lipid particle and/or lipid particle composition, to a subject in need of such treatment.

The lipid particle and/or lipid particle composition may be delivered to a subject as a component of a vaccine.

### BRIEF DESCRIPTION OF THE FIGURES

FIG. 1: shows the intensity values plotted as a function of pH using GraphPad Prism and fit with a sigmoidal dose response curve for SL01. The apparent pKa was determined as the EC50 of this curve, where half of the ionizable amines are expected to be protonated.

FIG. 2: shows the intensity values plotted as a function of pH using GraphPad Prism and fit with a sigmoidal dose response curve for SL02. The apparent pKa was determined as the EC50 of this curve, where half of the ionizable amines are expected to be protonated.

FIG. 3: shows both SL01 and SL02 are able to transfect BHK-V cells in vitro. SL02 LNPs trended higher in potency for both saRNA constructs examined.

FIG. 4: shows LNPs encapsulating saRNA expressing influenza antigens H5 and N1 led to high levels of neutralizing antibodies in mice as measured by a hemagglutination inhibition (HAI) assay following a prime-boost vaccination schedule. At a 0.1 ug saRNA dose, the values for both LNPs were comparable to those elicited by an inactivated virus vaccine adjuvanted with MF59. Dotted line is the limit of detection.

FIG. 5: shows LNPs encapsulating saRNA expressing influenza antigens H5 and N1 led to high levels of neutralizing antibodies in mice as measured by a pseudovirus microneutralization (MN) assay following a prime-boost vaccination schedule. At a 0.1 ug saRNA dose, the values for both LNPs were comparable to those elicited by an inactivated virus vaccine adjuvanted with MF59. Dotted line is the limit of detection.

FIG. 6: shows SL01 and SL02 LNPs encapsulating saRNA expressing influenza antigens H5 and N1 led to measurable levels of activated antigen-specific CD8 T cells following a prime-boost vaccination schedule in contrast to an inactivated virus vaccine adjuvanted with MF59.

### DETAILED DESCRIPTION

The present disclosure is based on the use of a lipid nanoparticle (LNP) and/or LNP composition, wherein the LNPs comprise certain ionizable lipid compounds, one or more of Compounds 1-12 as defined herein, for the delivery of a messenger RNA. The ionizable lipid compounds present biodegradable groups which may assist in reducing toxicity or improving clearance, in vivo.

In one embodiment, the LNP and/or LNP composition may be a component of a vaccine although the therapeutic use of the LNP and/or LNP compositions described herein is not so limited.

In some embodiments the LNP and/or LNP composition may be suitable for the delivery of messenger RNA for expression of a protein encoded by the messenger RNA.

In some embodiments, the LNP and/or LNP composition may be suitable for the delivery of a messenger RNA as a component of a messenger RNA vaccine.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the present disclosure.

Any embodiment of the present disclosure shall be taken to apply mutatis mutandis to any other embodiment of the disclosure unless specifically stated otherwise.

Unless specifically defined otherwise, all technical and scientific terms used herein shall be taken to have the same meaning as commonly understood by one of ordinary skill in the art (for embodiments, in organic synthetic chemistry, cell culture, molecular genetics, immunology, immunohistochemistry, protein chemistry, and biochemistry).

Unless otherwise indicated, any recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The terms “from” and “to”, when indicating a range, shall be understood to mean the range is inclusive of the recited lower and upper values. For example, “x is an integer from 0 to 6” shall be understood as including the situation in which x is not present (x is 0), that in which x is 6, as well as each whole number integer value in between, i.e. x is 1, 2, 3, 4, or 5.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

As used herein, the term “lipid nanoparticle” or “LNP” shall be understood to refer to lipid-based particles having at least one dimension on the order of nanometers (e.g., 1-1,000 nm) and which comprises a compound selected from Compounds 1-12 described herein. In embodiments, LNPs are formulated in a composition for delivery of a messenger RNA, including a conventional messenger RNA or a self-amplifying messenger RNA to a desired target such as a cell, tissue, organ, tumor, and the like. The LNPs comprise an ionizable cationic compound selected from one or more of Compounds 1-12 and one or more of a neutral lipid, charged lipid, sterol and PEGylated lipid.

A “cationic compound”, “ionizable cationic compound”, “cationic lipid compound”, “ionizable cationic lipid compound”, “ionizable lipid compound” or like terms, refers to lipid compounds selected from Compounds 1-12, as described herein, which are capable of bearing a positive charge. Ionizable cationic lipids disclosed herein include one or more nitrogen-containing groups which may bear the positive charge. They are ionizable such that they can exist in a positively charged or neutral form, depending on pH. The ionization of the cationic lipid may affect the surface charge of the lipid nanoparticle under different pH conditions.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and phophatidylethanolamines such as 1,2-Dioleoyl-sn-glycero-3-phosphoethanolamine (DOPE), and sphingomyelins (SM).

The term “charged lipid” refers to any of a number of lipid species that exist in either a positively charged or negatively charged form independent of the pH within a useful physiological range e.g. pH˜3 to pH˜9. Non-limiting examples of charged lipids include phosphatidylserines, phosphatidic acids, phosphatidylglycerols, phosphatidylmositols, sterol hemisuccinates, dialkyl trimethylammonium-propanes, (including DOTAP and DOTMA), dialkyl dimethylaminopropanes, ethyl phosphocholines, and dimethylaminoethane carbamoyl sterols.

The term “messenger RNA” as used herein refers to a conventional, non-replicating messenger RNA (conventional mRNA) as well as a self-amplifying messenger RNA (saRNA), and combinations thereof. The term refers generally to a polynucleotide that encodes at least one polypeptide. Messenger RNA may contain one or more coding and non-coding regions. It may be purified from natural sources, produced using recombinant expression systems and optionally purified, chemically synthesized, and the like. The messenger RNA may comprise polynucleotides containing known nucleotide analogs or modified backbone residues or linkages, which are synthetic, naturally occurring, and non-naturally occurring, and which have similar binding properties as the reference polynucleotide. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses messenger RNAs including polynucleotides containing known analogues of natural nucleotides that have similar binding properties as the reference polynucleotide. Unless otherwise indicated, a particular messenger RNA nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “expression” as used herein refers to translation of a messenger RNA into a polypeptide, the assembly of polypeptides into an intact protein and/or post-translational modification of a polypeptide or fully assembled protein.

An “effective amount” or “therapeutically effective amount” of a therapeutic messenger RNA is an amount sufficient to produce the desired effect, such as an increase in expression of a target polypeptide in comparison to the normal expression level detected in the absence of the messenger RNA. Suitable assays for measuring expression of a target gene or target sequence include, examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays.

As used herein, “prodrug” is meant to indicate a compound that may be converted under physiological conditions or by solvolysis to any of Compounds 1-12 as described herein. Thus, the term “prodrug” refers to a metabolic precursor of such a compound that is pharmaceutically acceptable. A prodrug may be inactive when administered to a subject in need thereof, but is converted in vivo to an active form. The term may also include any covalently bonded carriers, which release the active compound in vivo when such a prodrug is administered to a mammalian subject. Prodrugs of any of Compounds 1-12 may be prepared by modifying functional groups present in the compound in such a way that the modifications are cleaved, either in routine manipulation or in vivo, to the parent compound.

“Pharmaceutically acceptable carrier, diluent or excipient”, or like terms, refers to any ingredient other than Compounds 1-12 described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxy toluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

“Pharmaceutically acceptable salt” includes both acid and base addition salts. Lists of suitable salts may be found in Remington's Pharmaceutical Sciences, 18th ed., Mack Publishing Company, Easton, PA, 1990, p. 1445, and Journal of Pharmaceutical Science, 66, 2-19 (1977). Acid addition salts are those which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like. Base addition salts are those which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

As used herein, the term “biodegradable group” is a group that may facilitate faster metabolism of a lipid in a mammal. An ester is a suitable biodegradable group and Compounds 1-12 of the present disclosure present two such ester groups for improved in vivo biodegradability while Compound 10 additionally presents disulfide bonds which may be similarly biodegradable via an intracellular reduction step.

As used herein, “encapsulation efficiency” refers to the amount of a messenger RNA that becomes part of an LNP composition, relative to the initial total amount of messenger RNA used in the preparation of the LNP composition. For example, if 92 mg of messenger RNA are encapsulated in an LNP composition out of a total 100 mg of messenger RNA initially provided to the composition, the encapsulation efficiency may be given as 92%. As used herein, “encapsulation” may refer to complete, substantial, or partial enclosure, confinement, surrounding, or encasement.

As used herein, the term “subject” shall be taken to mean any animal, such as a mammal, and including humans. Exemplary subjects include but are not limited to humans and non-human primates. For example, the subject is a human.

As used herein, the term “mammal” includes humans and both domestic animals such as laboratory animals and household pets (e.g., cats, dogs, swine, cattle, sheep, goats, horses, rabbits), and non-domestic animals such as wildlife and the like.

### Compounds

In embodiments, the compounds selected from Compounds 1-12, below, may provide for advantages over other select prior art ionizable cationic lipid compounds in a messenger RNA-LNP including one or more of: improved complexation with a messenger RNA; beneficial pKa properties; improved messenger RNA encapsulation efficiency as part of an LNP; reduced toxicity; improved biodegradability; improved in vivo clearance; desirable N:P ratio when complexed with a messenger RNA; desirable polydispersity index for the LNPs comprising them; and improved LNP formation.

In one broad form, the present disclosure provides for a lipid nanoparticle associated with a messenger RNA, the lipid nanoparticle comprising one or more ionizable cationic lipid compounds selected from the group consisting of:

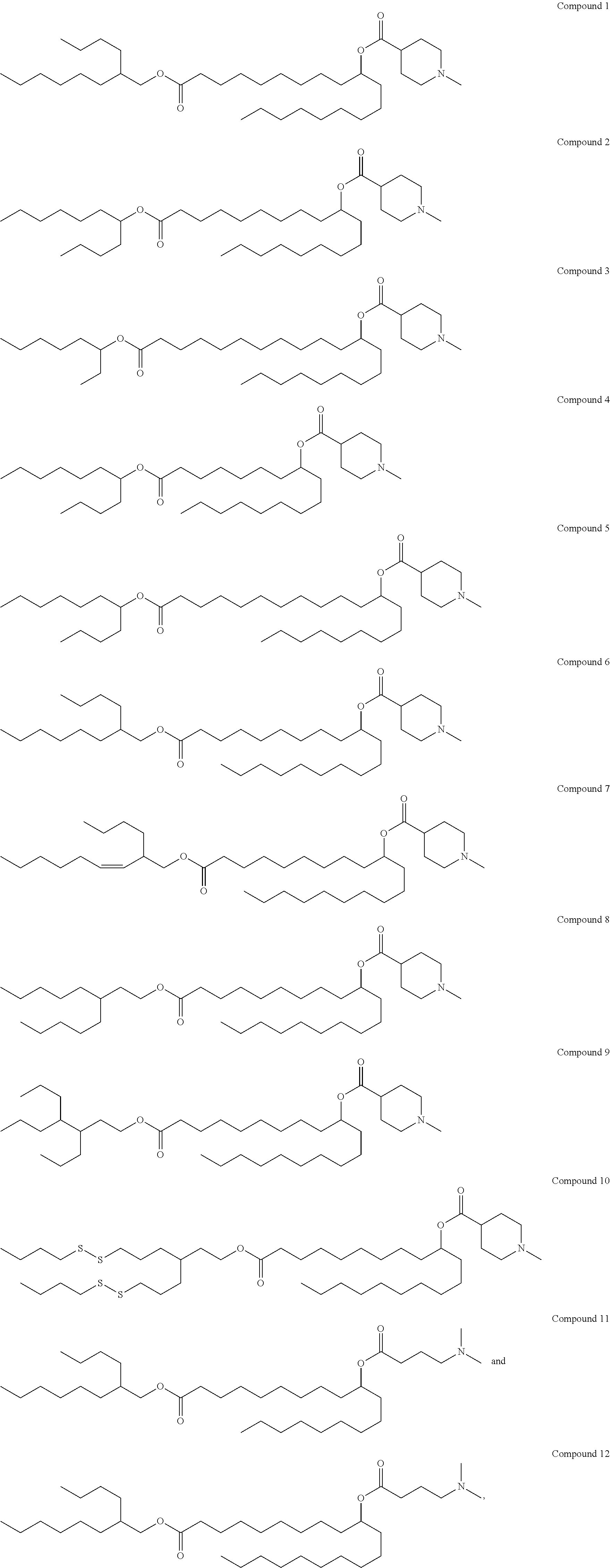

or a pharmaceutically acceptable salt or prodrug thereof.

### Lipid Nanoparticles

The present disclosure provides for an LNP associated with a messenger RNA, wherein the LNP comprises a compound selected from the group consisting of Compounds 1-12, as described herein, such as a compound selected from the group consisting of Compounds 2-12.

In embodiments, the LNP comprises a messenger RNA.

In embodiments, the LNP encapsulates the messenger RNA.

In embodiments, the LNPs have a mean diameter of from about 30 nm to about 160 nm, from about 40 nm to about 160 nm, from about 50 nm to about 160 nm, from about 60 nm to about 160 nm, from about 70 nm to about 160 nm, from about 50 nm to about 140 nm, from about 60 nm to about 130 nm, from about 70 nm to about 120 nm, from about 80 nm to about 120 nm, from about 90 nm to about 120 nm, from about 70 to about 110 nm, from about 80 nm to about 110 nm, or about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm or 160 nm. The diameter of the LNP may be measured by dynamic light scattering (DLS), transmission electron microscopy (TEM), scanning electron microscopy (SEM), or other methods such as are known in the art.

In some embodiments, the LNPs may be relatively homogenous. A polydispersity index may be used to indicate the homogeneity of the LNPs. A small, for example less than 0.3 or less than 0.2, polydispersity index generally indicates a narrow particle size distribution. A composition of the LNPs described herein may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the LNP composition may be from about 0 to about 0.20 or 0.05 to 0.20.

The LNP may comprise one or more different compounds selected from Compounds 1-12, as appropriate. The inclusion of more than one such compound may, for example, be employed to achieve a desired pKa profile.

The LNP may comprise a compound selected from Compounds 1-12 and an additional cationic and/or ionizable lipid, for example a cationic and/or ionizable lipid comprising a cyclic or non-cyclic amine. Such additional cationic and/or ionizable lipids may be selected from the non-limiting group consisting of:
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino) butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)),
(2S)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2S)),
((4-hydroxybutyl) azanediyl)bis(hexane-6,1-diyl)bis(2-hexyldecanoate)) and
8-[(2-hydroxyethyl) [6-oxo-6-(undecyloxy) hexyl]amino]-octanoic acid, 1-octylnonyl ester.

In embodiments, the LNP additionally comprises one or more of a PEG-lipid, a sterol structural lipid and/or a neutral lipid.

### PEGylated Lipids

In one embodiment, the present disclosure provides an LNP comprising a compound selected from Compounds 1-12 and a PEGylated lipid, and the LNP associated with a messenger RNA.

It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For embodiment, a PEG lipid includes PEG-c-DOMG, PEG-DMG, PEG-DLPE, PEG-DMPE, PEG-DPPC, a PEG-DSPE lipid and combinations thereof.

### Neutral Lipids

In one embodiment, the present disclosure provides an LNP comprising a compound selected from Compounds 1-12 and a neutral lipid, and the LNP associated with a messenger RNA.

Suitable neutral or zwitterionic lipids for use in the present disclosure will be apparent to the skilled person and include, in embodiments, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

### Structural Lipids

In one embodiment, the present disclosure provides an LNP comprising a compound selected from Compounds 1-12 and a structural lipid, and the LNP associated with a messenger RNA.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol.

In one embodiment, the structural lipid is a sterol. In embodiments, the structural lipid is cholesterol. In another embodiment, the structural lipid is campesterol.

In embodiments, the LNPs comprise an ionizable cationic lipid compound selected from Compounds 1-12; a neutral lipid; a sterol such as cholesterol; and a PEGylated lipid. The LNPs are formulated with a messenger RNA to be delivered to a subject.

In certain embodiments, the LNPs comprise an ionizable cationic lipid compound selected from Compounds 1-10; a neutral lipid; a sterol such as cholesterol; and a PEGylated lipid. The LNPs are formulated with a messenger RNA to be delivered to a subject.

In embodiments, a plurality of the LNPs form an LNP composition as described herein.

### Messenger RNA

One or more compounds selected from Compounds 1-12, as described herein, may form complexes with, and so be formulated into LNPs with, a range of messenger RNA molecules and including both conventional or non-replicating messenger RNAs (referred to herein as conventional mRNA) and self-amplifying messenger RNAs (referred to herein as saRNAs). In this manner the LNPs and compositions may, in some embodiments, be used to induce expression of a desired protein both in vitro and in vivo by contacting cells with an LNP comprising one or more of Compounds 1-12, wherein the LNP encapsulates or is associated with a messenger RNA that is expressed to produce the desired protein.

Therefore, in embodiments, the messenger RNA is a conventional mRNA or saRNA encoding a polypeptide of interest, including any naturally or non-naturally occurring or otherwise modified polypeptide. A polypeptide encoded by a conventional mRNA or saRNA may be of any size and may have any secondary structure or activity. In some embodiments, a polypeptide encoded by a conventional mRNA or saRNA may have a therapeutic effect when expressed in a cell.

Messenger RNA useful for formulation with the LNPs incorporating an ionizable cationic compound selected from Compounds 1-12 may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a messenger RNA further includes a poly-A region or a Kozak sequence (e.g., in the 5′-UTR). In some cases, messenger RNAs may contain one or more intronic sequences capable of being excised from the messenger RNA. In some embodiments, a messenger RNA may include a 5′ cap structure, a chain terminating nucleotide, a stem loop, a poly A sequence, and/or a polyadenylation signal. Any one of the regions of a messenger RNA may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxy uridine), a 1-substituted pseudouridine (e.g., 1-methyl-pseudouridine or 1-ethyl-pseudouridine), and/or a 5-substituted cytidine (e.g., 5-methyl-cytidine).

Messenger RNA useful for formulation with the LNPs incorporating an ionizable cationic compound selected from Compounds 1-12 may include an internal ribosome entry site (IRES). An IRES may act as a sole ribosome binding site, or as one of multiple ribosome binding sites. A messenger RNA containing more than one functional ribosome binding site may encode several peptides or polypeptides that are translated independently by the ribosomes. Suitable IRES sequences that may be useful include those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (S1V) oand cricket paralysis viruses (CrPV).

An exemplary messenger RNA useful for formulation with the LNPs incorporating an ionizable cationic compound selected from Compounds 1-12 include a first region of linked nucleosides encoding an antigenic polypeptide, a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region.

Messenger RNAs suitable for use with the present LNPs may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

In some embodiments, messenger RNAs may include one or more alternative components, as described herein, which impart useful properties including increased stability and/or the lack of a substantial induction of the innate immune response of a cell into which the poly messenger RNA is introduced. For example, an alternative messenger RNA exhibits reduced degradation in a cell into which the messenger RNA is introduced, relative to a corresponding unaltered messenger RNA. These alternative species may enhance the efficiency of protein production, intracellular retention of the messenger RNA, and/or viability of contacted cells, as well as possess reduced immunogenicity.

Messenger RNA nucleotides may be naturally or non-naturally occurring. The messenger RNA may include one or more modified (e.g., altered or alternative) nucleobases, nucleosides, nucleotides, or combinations thereof. The messenger RNA may include any useful modification or alteration, such as to the nucleobase, the sugar, or the internucleoside linkage (e.g., to a linking phosphate/to a phosphodiester linkage/to the phosphodiester backbone). In some embodiments, one or more alterations are present in each of the nucleobase, the sugar, and the internucleoside linkage.

Messenger RNA may or may not be uniformly altered along the entire length of the molecule. For example, one or more or all types of nucleotide (e.g., purine or pyrimidine, or any one or more or all of A, G, U, C) may or may not be uniformly altered in a messenger RNA, or in a given predetermined sequence region thereof.

Different sugar alterations and/or internucleoside linkages (e.g., backbone structures) may exist at various positions in a messenger RNA. One of ordinary skill in the art will appreciate that the nucleotide analogs or other alteration(s) may be located at any position(s) of a messenger RNA such that the function of the messenger RNA is not substantially decreased. An alteration may also be a 5′- or 3 ‘-terminal alteration. In some embodiments, the messenger RNA includes an alteration at the 3’-terminus.

### Nucleobase Alternatives

The alternative nucleosides and nucleotides can include an alternative nucleobase. A nucleobase of a messenger RNA is an organic base such as a purine or pyrimidine or a derivative thereof. A nucleobase may be a canonical base (e.g., adenine, guanine, uracil, thymine, and cytosine). These nucleobases can be altered or wholly replaced to provide messenger RNA molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m3U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nm5s2U), 5-methylaminomethyl-uracil (mnm5U), 5-methylaminomethyl-2-thio-uracil (mnm5s2U), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnm5s2U), 5-propynyl-uracil, 1-propynyl-pseudouracil, 5-taurinomethyl-uracil (tm5U), 1-taurinomethyl-pseudouridine, 5-taurinomethyl-2-thio-uracil (tm5s2U), 1-taurinomethyl-4-thio-pseudouridine, 5-methyl-uracil (m5U, i.e., having the nucleobase deoxythymine), 1-methyl-pseudouridine (mψ), 1-ethyl-pseudouridine (Et1ψ), 5-methyl-2-thio-uracil (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl) uracil (acp3ψ), 1-methyl-3-(3-amino-3-carboxypropyl) pseudouridine (acp3 ψ), 5-(isopentenylaminomethyl) uracil (inm5U), 5-(isopentenylaminomethyl)-2-thio-uracil (inm5s2U), 5,2′-O-dimethyl-uridine (m5Um), 2-thio-2′-O_methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil. In one embodiment, the modified uracil is pseudouridine. In one embodiment, the modified uracil is N1-methyl-pseudouridine.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine. In one example, the modified cytosine is 5-methyl-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (16A), 2-methylthio-N6-isopentenyl-adenine (ms216A), N6-(cis-hydroxyisopentenyl) adenine (io6A), 2-methylthio-N6-(cis-hydroxyisopentenyl) adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6, N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mll), wyosine (imG), methylwyosine (mimG), 4-demethyl-wyosine (imG-14), isowyosine (imG2), wybutosine (yW), peroxywybutosine (02yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQ0), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

Conventional mRNA for formulation with LNPs comprising a compound selected from one or more of Compounds 1-12 may be prepared according to any available technique known in the art. Messenger RNA may be prepared by, for example, enzymatic synthesis which provides a process of template-directed synthesis of RNA molecules from an engineered DNA template comprised of an upstream bacteriophage promoter sequence linked to a downstream sequence encoding the gene of interest. Template DNA can be prepared for in vitro transcription from a number of sources with appropriate techniques which are well-known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N. Y. Humana Press, 2012)

Transcription of the RNA occurs in vitro using the appropriate linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well-known in the art (see, e.g. Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed messenger RNA is then purified from the undesired components of the transcription or associated reactions. Techniques for the isolation of the messenger RNA transcripts are well known in the art and include phenol/chloroform extraction or precipitation with either alcohol in the presence of monovalent cations or lithium chloride.

In embodiments, the messenger RNA associated with the LNP is a self-amplifying messenger RNA (saRNA) molecule. In certain embodiments, the saRNA is derived from or based on an alphavirus. Such saRNA molecules are known in the art and can be produced using replication elements derived from, for example, alphaviruses substituting the structural viral proteins with a nucleotide sequence encoding a protein of interest. The target cell to which the saRNA is delivered generates an exponential increase of encoded gene products, such as proteins or antigens, which can accumulate in the cells or be secreted therefrom.

The saRNA may contain one or more genes selected from the group consisting of viral replicases, viral proteases, viral helicases and other nonstructural viral proteins, and may also comprise 5′- and 3′-end cis-active replication sequences and, optionally, a heterologous sequence that encodes a desired amino acid sequence. A subgenomic promoter that directs expression of the heterologous sequence may be present. In embodiments, the heterologous sequence may be fused in frame to other coding regions in the saRNA and/or be under the control of an internal ribosome entry site (IRES).

The saRNA can be directly translated after delivery to a target cell providing a RNA-dependent RNA polymerase which then produces transcripts from the delivered RNA thereby providing multiple RNA transcripts which are antisense relative to the delivered RNA.

In embodiments, the saRNA employs an alphavirus-based RNA replicon. Alphavirus-based replicons are (+)-stranded replicons that can be translated after delivery to a cell to provide a replicase (or replicase-transcriptase). The replicase is translated as a polyprotein which auto-cleaves to provide a replication complex which creates genomic (−)-strand copies of the (+)-strand delivered RNA. These (−)-strand transcripts can themselves be transcribed to give further copies of the (+)-stranded parent RNA and also to give a subgenomic transcript which encodes the desired gene product. Translation of the subgenomic transcript thereby leads to in situ expression of the desired gene product by the transfected cell. Suitable alphavirus replicons can use a replicase from, for example, a sindbis virus, a semliki forest virus, an eastern equine encephalitis virus, a Venezuelan equine encephalitis virus, and the like.

A preferred saRNA molecule therefore encodes (i) a RNA-dependent RNA polymerase which can transcribe RNA from the saRNA molecule and (ii) a polypeptide antigen. The polymerase can be an alphavirus replicase, for example comprising alphavirus protein nsP4.

In embodiments, a suitable saRNA may have two open reading frames. The first (5′) open reading frame encodes a replicase; the second (3′) open reading frame encodes a polypeptide antigen. In some embodiments the RNA may have additional (downstream) open reading frames e.g. that encode another desired gene product. A suitable saRNA molecule can have a 5′ sequence which is compatible with the encoded replicase.

In embodiments, the saRNA may comprise one or more modified nucleotides as defined above.

The saRNA of the invention may encode one or more polypeptide antigens that contain a range of epitopes such as epitopes capable of eliciting either a helper T-cell response or a cytotoxic T-cell response, or both.

In embodiments, the saRNA may be engineered to express multiple nucleotide sequences, from two or more open reading frames, thereby allowing co-expression of proteins, such as two or more antigens together with cytokines or other immunomodulators, which can enhance the generation of an immune response. Such a saRNA molecule might be useful in the simultaneous production of various proteins as a bivalent or multivalent vaccine.

The saRNA may be prepared using any suitable method known in the art. A saRNA molecule that contains modified nucleotides can be prepared by transcribing a DNA that encodes the saRNA molecule using a suitable DNA-dependent RNA polymerase, such as T7 phage RNA polymerase, SP6 phage RNA polymerase, T3 phage RNA polymerase, and the like, or mutants of the polymerases which allow efficient incorporation of modified nucleotides into RNA. The incorporation of nucleotide analogs into a saRNA may be employed to alter the stability of such RNA molecules, to increase resistance against RNases, to establish replication after introduction into appropriate host cells (“infectivity” of the RNA), and/or to induce or reduce innate and adaptive immune responses.

In embodiments, the saRNA can be screened to confirm their therapeutic and prophylactic properties using various in vitro or in vivo testing methods that are known to those of skill in the art. For example, vaccines comprising saRNA can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-amplifying messenger RNA molecule that encodes a polypeptide antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

In embodiments, saRNA that encodes a polypeptide antigen can be tested for the ability to induce humoral immune responses, as evidenced, for example, by induction of B cell production of antibodies specific for an antigen of interest. These assays can be conducted using, in one embodiment, peripheral B lymphocytes from immunized individuals.

### Lipid Nanoparticle Formation

LNPs associated with a messenger RNA and comprising a compound selected from one or more of Compounds 1-12 can be made using approaches which are well-known in the art of formulation. For example, suitable LNPs can be formed using mixing processes such as microfluidics, including herringbone micromixing, and T-junction mixing of two fluid streams, one of which contains the messenger RNA, typically in an aqueous solution, and the other of which has the various required lipid components, typically in ethanol.

The LNPs may then be prepared by combining a compound selected from one or more of Compounds 1-12, a phospholipid (such as DOPE or DSPC, which may be purchased from commercial sources including Avanti Polar Lipids, Alabaster, AL), a PEGylated lipid (such as 1,2-dimyristoyl-sn-glycerol methoxypoly ethylene glycol, also known as PEG-DMG, which may be purchased from commercial sources including Avanti Polar Lipids, Alabaster, AL), and a structural lipid/sterol (such as cholesterol, which may be purchased from commercial sources including Sigma-Aldrich), at concentrations of, for example, about 50 mM in ethanol. Solutions should be refrigerated during storage at, for example, −20° C. The various lipids may be combined to yield the desired molar ratios and diluted with water and ethanol to a final desired lipid concentration of, for example, between about 5.5 mM and about 25 mM.

An LNP composition comprising a messenger RNA is prepared (as set out in the examples) by combining the above lipid solution with a solution including the messenger RNA at, for example, a lipid component to messenger RNA wt:wt ratio from about 5:1 to about 50:1. The lipid solution may be rapidly mixed with the messenger RNA solution using a NanoAssemblr microfluidic system at flow rates between about 3 ml/min and about 18 ml/min to produce a suspension with a water to ethanol ratio between about 1:1 and about 4:1, or between about 2:1 and about 4:1.

For LNP compositions including a messenger RNA, solutions of the messenger RNA at concentrations of 1.0 mg/ml in deionized water may be diluted in 50 mM sodium citrate buffer at a pH between 3 and 6 to form a stock solution.

LNP compositions may be further processed, as is known in the art, by 10-fold dilution into 50 mM citrate buffer at pH 6 and subjected to tangential flow filtration (TFF) using a 300 k molecular weight cut-off membrane (mPES) until concentrated to the original volume. Subsequently, in one example, the citrate buffer may be replaced with a buffer containing 20 mM Tris buffer at pH 7.5, 80 mM sodium chloride, and 3% sucrose using diafiltration with a 10-fold volume of the new buffer. The LNP solution may be concentrated to a volume of, for example, between 5-10 mL, filtered using a 0.2 micron PES syringe filter, aliquoted into vials, and frozen at 1° C./min using a Corning® CoolCell® LX Cell Freezing Container until the samples reach −80° C. Samples may be stored at −80° C. until needed.

The method described above induces nano-precipitation and particle formation. Alternative processes including, but not limited to, T-junction and direct injection, may be used to achieve the same nano-precipitation.

In some embodiments, the lipid component of the LNP formulation comprises about 25 mol % to about 60 mol % of a Compound of structures 1-12, about 2 mol % to about 25 mol % phospholipid (neutral lipid), about 18.5 mol % to about 60 mol % structural lipid (sterol), and about 0.2 mol % to about 10 mol % of PEGylated lipid, provided that the total mol % does not exceed 100%. In some embodiments, the lipid component of the LNP formulation comprises about 30 mol % to about 50 mol % of a Compound of structures 1-12, about 5 mol % to about 20 mol % phospholipid, about 30 mol % to about 55 mol % structural lipid, and about 1 mol % to about 5 mol % of PEGylated lipid. In a particular embodiment, the lipid component includes about 40 mol % of a Compound of structures 1-12, about 10 mol % phospholipid, about 48 mol % structural lipid, and about 2.0 mol % of PEG lipid. In some embodiments, the phospholipid may be DOPE or DSPC. In other embodiments, the PEG lipid may be PEG-DMG and/or the structural lipid may be cholesterol.

The efficiency of encapsulation of the messenger RNA within the LNPs comprising a compound selected from Compounds 1-12 may be at least 50%, for example about 50%, 55%, 60%, 65%, 70%, 75%, 80%, 85%, 90%, 91%, 92%, 93%, 94%, 95%, 96%, 97%, 98%, or 99%. In some embodiments, the encapsulation efficiency may be at least 80%. In certain embodiments, the encapsulation efficiency may be at least 90%.

### Lipid Nanoparticle Compositions

The LNPs comprising a compound selected from Compounds 1-12 and a messenger RNA can be formulated for administration via any accepted mode of administration of lipid particles including LNPs, liposomes, lipid vesicles and like lipid-based particles. The pharmaceutical compositions of the invention may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical LNP compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. The compositions administered to a subject may be in the form of one or more dosage units, where for example, a tablet or injectable liquid volume may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000).

Therefore, one embodiment of the present disclosure provides a composition (such as a pharmaceutical composition) comprising an LNP, which comprises a compound selected from Compounds 1-12, combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for embodiment described in Remington's Pharmaceutical Sciences (Mack Publishing Co. N.J. USA, 1991).

An LNP is useful for parenteral, topical, oral, or local administration, intramuscular administration, aerosol administration, or transdermal administration, for prophylactic or for therapeutic treatment. In one embodiment, the LNP is administered parenterally, such as intramuscularly, subcutaneously or intravenously. In some embodiments, the LNP is administered intramuscularly.

Formulation of LNPs to be administered will vary according to the route of administration and formulation (e.g., solution, emulsion, capsule) selected. An appropriate pharmaceutical composition comprising an LNP to be administered can be prepared in a physiologically acceptable carrier. For solutions or emulsions, suitable carriers include, for embodiment, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for embodiment, sodium acetate, sodium chloride, potassium chloride, calcium chloride and sodium lactate. The LNPs can be stored in the liquid stage or can be lyophilized for storage and reconstituted in a suitable carrier prior to use according to art-known lyophilization and reconstitution techniques.

When the LNP composition is a vaccine composition then the carrier may be water, typically pyrogen-free water; isotonic saline or buffered (aqueous) solutions, e.g phosphate, citrate etc. buffered solutions. For injection of an LNP vaccine composition, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, such as at least 3 mM of a potassium salt. In an embodiment, the sodium, calcium and, optionally, potassium salts may be present as their chlorides, iodides, or bromides, or in the form of their hydroxides, carbonates, hydrogen carbonates, or sulfates, etc. Non-limiting examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. In certain embodiments, the buffer suitable for injection purposes, may contain salts selected from sodium chloride (NaCl), calcium chloride (CaCl₂)) and optionally potassium chloride (KCl), wherein further anions may be present additional to the chlorides. In embodiments, the salts in the injection buffer are present in a concentration of at least 50 mM sodium chloride (NaCl), at least 3 mM potassium chloride (KCl) and at least 0.01 mM calcium chloride (CaCl₂). The injection buffer may be hypertonic, isotonic or hypotonic with reference to the specific reference medium.

In some embodiments of a vaccine, one or more compatible solid or liquid fillers or diluents or encapsulating compounds may be employed which are suitable for administration to a person. Pharmaceutically acceptable carriers, fillers and diluents will have sufficiently high purity and sufficiently low toxicity to make them suitable for administration to a subject. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; and alginic acid.

When the LNP composition is a vaccine composition it may further comprise one or more pharmaceutically acceptable adjuvants to enhance the immunostimulatory properties of the composition. The adjuvant may be any compound, which is suitable to support administration and delivery of the LNP composition and which may initiate or increase an immune response of the innate immune system, i.e. a non-specific immune response.

Such an adjuvant may be selected from any adjuvant known to a skilled person and suitable for the particular nature of the vaccine, i.e. for induction of a suitable immune response in a mammal. In embodiments, the adjuvant may be selected from the group consisting of: MF59R (squalene-water emulsion), TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii)dimethyldioctadecylammonium chloride (DDA), iii) zinc-L-proline salt complex (ZnPro-8); GM-CSF); GMDP (N-acetylglucosaminyl-(bl-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-lbeta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, AL); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thrl]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG polynucleotide sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide. In one preferred example the adjuvant may be the oil-in-water emulsion adjuvant MF59R, particularly if the vaccine is an influenza vaccine.

Upon formulation, compositions of the present disclosure will be administered in a manner compatible with the dosage formulation and in such amount as is therapeutically/prophylactically effective. The dosage ranges for the administration of the LNPs are those large enough to produce the desired effect. For embodiment, the composition comprises an effective amount of the encapsulated or associated messenger RNA, i.e., the conventional mRNA or saRNA. In one embodiment, the composition comprises a therapeutically effective amount of the messenger RNA. In another embodiment, the composition comprises a prophylactically effective amount of the messenger RNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Preparation methods for the above compounds and compositions are described further herein and/or are known in the art.

### Methods of Treatment and Producing a Polypeptide of Interest

Diseases, disorders, and/or conditions which may be a result of or related to aberrant protein or polypeptide production may be treated by the present LNPs comprising a messenger RNA and a compound selected from Compounds 1-12 and may include, but are not limited to, rare diseases, infectious diseases, cancer and proliferative diseases, genetic diseases, autoimmune diseases, diabetes, neurodegenerative diseases, cardio- and reno-vascular diseases, and metabolic diseases.

In embodiments, the messenger RNA, including conventional mRNA and saRNA, component of the LNPs may be used to express a polypeptide of interest. Certain diseases in humans are caused by the absence or impairment of a functional protein in a cell type where the protein is normally present and active. The functional protein can be completely or partially absent due, for example, to transcriptional inactivity of the encoding gene or due to the presence of a mutation in the encoding gene that renders the protein completely or partially non-functional.

LNP compositions may be formulated in dosage unit form. The therapeutically effective or prophylactically effective dose for any particular patient will depend upon a variety of factors including the severity and identify of a disorder being treated; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well-known in the medical arts.

LNP compositions described herein may be used in combination with one or more other therapeutic, prophylactic, diagnostic, or imaging agents. They may be administered together in a single composition or administered separately in different compositions.

The present disclosure provides methods of producing a polypeptide of interest in a mammalian cell. Methods of producing polypeptides involve contacting a cell with an LNP composition, as described herein, comprising a messenger RNA encoding the polypeptide of interest. Upon contacting the cell with the LNP composition, the messenger RNA may be taken up and translated in the cell to produce the polypeptide of interest.

In some embodiments, a polypeptide of interest may be specifically produced in a target cell or tissue of interest. That is, the production of polypeptide is higher in the target cell or tissue than in a non-target cell or tissue.

The step of contacting an LNP composition including a messenger RNA with a cell may involve or cause transfection. One or more of the constituent LNP lipids, such as for example the neutral lipid (for example a phospholipid), the PEGylated lipid or the sterol may facilitate transfection and/or increase transfection efficiency, for example, by interacting and/or fusing with a cellular or intracellular membrane to promote translocation into the cellular cytosol where translation can occur. More specifically, transfection may occur by endosomal uptake of LNPs followed by endosomal destabilization and escape caused by interaction of the LNP lipid components and the endosomal membrane. Transfection may allow for the translation of the messenger RNA within the cell.

In some embodiments, the LNP compositions described herein may be used therapeutically. For example, a messenger RNA included in the LNP composition may encode a therapeutic polypeptide (e.g., in a translatable region) and produce the therapeutic polypeptide upon contacting and/or entry (e.g., transfection) into a cell. In other embodiments, a messenger RNA included in the LNP composition may encode a polypeptide that may improve or increase the immunity of a subject.

In embodiments, a messenger RNA included in an LNP composition may encode a recombinant polypeptide that may replace one or more polypeptides that may be substantially absent in a cell contacted with the LNP composition. The one or more substantially absent polypeptides may be lacking due to a genetic mutation of the encoding gene or a regulatory pathway thereof. Alternatively, a recombinant polypeptide produced by translation of the messenger RNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the messenger RNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the messenger RNA may be engineered for localization within the cell, such as within a specific compartment such as the nucleus, or may be engineered for secretion from the cell or for translocation to the plasma membrane of the cell.

In some embodiments, contacting a cell with an LNP composition of the present disclosure including a messenger RNA may reduce the innate immune response of a cell to an exogenous polynucleotide. A cell may be contacted with a first LNP composition including a first amount of a first exogenous messenger RNA including a translatable region and the level of the innate immune response of the cell to the first exogenous messenger RNA may be determined. Subsequently, the cell may be contacted with a second LNP composition including a second amount of the first exogenous messenger RNA, the second amount being a lesser amount of the first exogenous messenger RNA compared to the first amount. Alternatively, the second composition may include a first amount of a second exogenous messenger RNA that is different from the first exogenous messenger RNA. The steps of contacting the cell with the first and second LNP compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

In some embodiments, the present disclosure provides for the use of the LNPs comprising a messenger RNA and a compound selected from Compounds 1-12 in the manufacture of a medicament for the treatment of a disease, disorder or condition. The disease, disorder or condition may be as described in any one or more embodiments herein.

The medicament may be for the prevention or treatment of a cancer, an infectious disease, an allergy, or an autoimmune disease. In embodiments, the medicament is a vaccine. The vaccine may be a tumor vaccine, an influenza vaccine, or a SARS-CoV-2 vaccine.

### Use of LNPs in a Vaccine

In some embodiments, the LNP comprising a compound selected from Compounds 1-12 and a messenger RNA may be a component of a vaccine. Vaccines include compounds and preparations that are capable of providing immunity against one or more conditions related to infectious diseases and so may include messenger RNAs encoding infectious disease-derived antigens and/or epitopes. Vaccines also include compounds and preparations that direct an immune response against cancer cells and can include messenger RNAs encoding tumor cell derived antigens, epitopes, and/or neoepitopes.

In embodiments, the vaccine may be a conventional mRNA vaccine or saRNA vaccine and so the LNP comprising a compound selected from Compounds 1-12 encapsulates or is associated with a conventional mRNA or saRNA molecule which comprises a sequence encoding an antigenic peptide or protein, or a fragment, variant or derivative thereof.

In embodiments, the messenger RNA encodes for an antigen from an infectious agent.

The antigenic peptides or proteins may be pathogenic antigens, tumour antigens, allergenic antigens or autoimmune self-antigens. Such pathogenic antigens may be those derived from pathogenic organisms, in particular bacterial, viral or protozoological (multicellular) pathogenic organisms, which evoke an immunological reaction in a mammalian subject, such as a human. Pathogenic antigens may be surface antigens, for example proteins or fragments thereof, located at the surface of the virus or the bacterial or protozoological organism.

Pathogenic antigens of interest may include those derived from one or more of: Acinetobacter baumannii, Anaplasma genus, Anaplasma phagocytophilum, Ancylostoma braziliense, Ancylostoma duodenale, Area nobacteri urn haemolyticum, Ascaris lumbricoides, Aspergillus genus, Astroviridae, Babesia genus, Bacillus anthracis, Bacillus cereus, Bartonella henselae, BK virus, Blastocysts hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, QD prion, Clonorchis sinensis, Clostridium botulinum, Clostridium difficile, Clostridium perfringens, Clostridium perfringens, Clostridium spp, Clostridium tetani, Coccidioides spp, coronavi ruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 0157: H7, Oll 1 and 0104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In certain embodiments, relevant antigens may be derived from the pathogens selected from: Severe Acute Respiratory Syndrome (SARS), Severe Acute Respiratory Syndrome Coronavirus and Severe Acute Respiratory Syndrome Coronavirus 2 (SARS-CoV-1 and SARS-CoV-2), Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus.

In some embodiments, the relevant pathogenic antigen may be selected from: Outer membrane protein A OmpA, biofilm associated protein Bap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GalNAc lectin, protein CRT, 125 kDa immunodominant antigen, protein M17, adhesin ADH112, protein STIRP (Entamoeba histolytica, Amoebiasis); Major surface proteins 1-5 (MSPla, MSPlb, MSP2, MSP3, MSP4, MSP5), type IV secreotion system proteins (VirB2, VirB7, VirBll, VirD4) (Anaplasma genus, Anaplasmosis); protective Antigen PA, edema factor EF, lethal facotor LF, the S-layer homology proteins SLH (Bacillus anthracis, Anthrax); acranolysin, phospholipase D, collagen-binding protein CbpA (Area nobacteri urn haemolyticum, Area nobacteri urn haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa suarface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Peplp, GPI-anchored protein Gellp, GPI-anchored protein Crflp (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 11C5, 21B4, P29, variant erythrocyte surface antigen VESA1, Apical Membrane Antigen 1 AMA-1 (Babesia genus, Babesiosis); hemolysin, enterotoxin C, PXO1-51, glycolate oxidase, ABC-transporter, penicillin-binding protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocysts hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BONT/A3, BONT/B, BONT/C, BONT/D, BONT/E, BONT/F, BoNT/G, recombinant botulinum toxin F He domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-bnding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B lalB, trigger factor Tig, molecular chaperone DnaK, putative peptidyl-prolyl cis-trans isomerase SurA, lipoprotein Omp19, outer membrane protein MotY Ompl6, conserved outer membrane protein D15, malate dehydrogenase Mdh, component of the Type-IV secretion system (T4SS) VirJ, lipoprotein of unknown function BAB1_0187 (Brucella genus, Brucellosis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspA1, protein FspA2 (Campylobacter genus, Campylobacteriosis); glycolytic enzyme enolase, secreted aspartyl proteinases SAPI-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Pari, mucin-Associated Surface Proteins MPSP (Trypanosoma cruzi, Chagas Disease (American trypanosomiasis)); envelope glycoproteins (gB, gC, gE, gH, gl, gK, gL), (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, PmplO, Pmpll, Pmpl2, Pmpl3, Pmpl4, Pmp15, Pmpl6, Pmpl7, Pmpl8, Pmpl9, Pmp20, Pmp21), (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis protein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GalXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gpl5, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein ppl5, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vQD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DltA, lipoprotein Hip, major outer membrane protein OMP, outer membrane protein OmpA2 (Haemophilus ducreyi, Chancroid); aspartyl protease 1 Pepl, phospholipase B PLB, alpha-mannosidase 1 AMN1, glucanosyltransferase GEL1, urease URE, peroxisomal matrix protein Pmp1, proline-rich antigen Pra, humal T-cell reative protein TorP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri tl, protein IV, glycerol-3-phosphate dehydrogenase Gpdl, osmosensor HwSholA, osmosensor HwSholB, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fmsl (EbpA(fm), Fms5 (EbpB(fm), Fms9 (EpbC(fm) and FmslO, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adrl (RP827), cell surface protein Adr2 (RP828), cell surface protein SCA1, Invasion protein invA, cell division protein fts, secretion proteins sec Ofamily, virulence proteins virB, tlyA, tlyC, parvulin-like protein Pip, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gpllO, glycoprotein gp42, glycoprotein gHgL, glycoprotein gB (Epstein-Barr Virus (EBV), Epstein-Barr Virus Infectious Mononucleosis); cpasid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins LI and L2, Kunitz-type protein KTM, leucine aminopeptidase LAP, cysteine proteinase Fas2, saposin-like protein-2 SAP-2, thioredoxin peroxidases TPx, Prx-1, Prx-2, cathepsin I cysteine proteinase CL3, protease cathepsin L CLI, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component 1b, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Decl (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alphal-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emml, M protein type 6 Emm6, immunoglobulin-binding protein 35 Sib35, Surface protein R28 Spr28, superoxide dismutase SOD, C5a peptidase ScpA, antigen I/II Agl/II, adhesin AspA, G-related alpha2-macroglobulin-binding protein GRAB, surface fibrillar protein M5 (Streptococcus pyogenes, Group A streptococcal infection); C protein β antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alphalike protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein PI, outer membrane protein P2, outer membrane lipoprotein Pep, Lipoprotein E, outer membrane protein P4, fuculokinase fucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein CpnIO, protein groES, heat shock protein HsplO, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, O-antigen, toxin-antigens Stx2B, toxin-antigen StxlB, adhesion-antigen fragment Int28, protein EspA, protein EspB, Intimin, protein Tir, protein IntC300, protein Eae (Escherichia coli 0157: H7, Olll and 0104: H4, Hemolytic-uremic syndrome (HUS)); RNA polymerase L, protein L, glycoprotein Gn, glycoprotein Gc, nucleocapsid protein S, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein PIB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35, envelope glycoprotein E2 NS1 gp68 gp70, capsid protein C, core protein Core, polyprotein, virus protein VP1, virus protein VP2, virus protein VP3, virus protein VP4, antigen G, protein NS3, protein NS5A, (Hepatitis C Virus, Hepatitis C); virus protein VP1, virus protein VP2, virus protein VP3, virus protein VP4, large hepaptitis delta antigen, small hepaptitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RSI), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein HIC, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, aspartic protease APR-1, acetylcholinesterase AChE (Ancylostoma duodenale and Necator americanus, Hookworm infection); protein NS1, protein NP1, protein VP1, protein VP2, protein VP3 (Human bocavirus (HBOV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSPla, MSPlb, MSP2, MSP3, MSP4, MSP5), type IV secreotion system proteins VirB2, VirB7, VirBll, VirD4 (Anaplasma phagocytophilum, Human granulocytic anaplasmosis (HGA)); protein NS1, small hydrophobic protein NS2, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein E5, protein E6, protein E7, protein E8, major capsid protein LI, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein Bl, ribosomal protein P1-like protein PI, sterol 24-c-methy transferase SMT, LACK protein, histone HI, SPB 1 protein, thiol specific antioxidant TSA, protein antigen STI1, signal peptidase SP, histone H2B, suface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membran lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol-specific phospholipase C PLC (PlcA, PlcB), O-acetyltransferase Oat, ABC-transporter permease Im.G_1771, adhesion protein LAP, LAP receptor Hsp60, adhesin LapB, haemolysin listeriolysin OLLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein Z, polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbeta merozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LolC, OppA, and PotF), putative lipoprotein releasing system transmembrane protein LolC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar ot integral membrane protein YIPA, a nti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RP07 (Molluscum contagiosum virus (MCV), Molluscum contagiosum (MC)); matrix protein M, phosphoprotein P/V, small hydrophobic protein SH, nucleoprotein N, protein V, fusion glycoprotein F, hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin PI, adhesion P30, protein pl16, protein P40, cytoskeletal protein HMW1, cytoskeletal protein HMW2, cytoskeletal protein HMW3, MPN152 coding protein, MPN426 coding protein, MPN456 coding protein, MPN-500 coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gpO, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PlpE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein Tbpl, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Prn, protein Fim2, protein Fim3; “(Bordetella pertussis, Pertussis (Whooping cough));” F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YPO0612, heat shock protein 90 HtpG, putative sulfatase protein YdeN, outer-membrane lipoprotein carrier protein LolA, secretion chaperone YerA, putative lipoprotein YPO0420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla;” (Yersinia pestis, Plague); protein PhpA, surface adhesin PsaA, pneumolysin Ply, ATP-dependent protease CIp, lipoate-protein ligase LplA, cell wall surface anchored protein psrP, sortase SrtA, glutamyl-tRNA synthetase GltX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfal, exendin-3, secretory lipase, cathepsin B-like protease, cysteine protease, cathepsin, peroxiredoxin, protein CrylAc (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, PmplO, Pmpll, Pmpl2, Pmpl3, Pmpl4, Pmp15, Pmpl6, Pmpl7, Pmpl8, Pmpl9, Pmp20, Pmp21) (Chlamydophila psittaci, Psittacosis); outer membrane protein PI, heat shock protein B HspB, peptide ABC transporter, GTP-binding protein, protein IcmB, ribonuclease R, phosphatas SixA, protein DsbD, outer membrane protein TolC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, nonstructural glycoprotein 4 NS4, outer capsid protein VP4; (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASA1, glutathione S-transferases GST, cystein protease, serine protease, apolipoprotein (Sarcoptes scabiei, Scabies); glutathione S-transferases GST, paramyosin, hemoglbinase SM32, major egg antigen, 14 kDa fatty acid-binding protein Sml4, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tri partite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein CI, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomysin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpFl, repeat protein Tpr, repeat protein F TprF, repeat protein G TprG, repeat protein I Tprl, repeat protein J TprJ, repeat protein K TprK, treponemal membrane protein A TmpA, lipoprotein, 15 kDa Tpp15, 47 kDa membrane antigen, miniferritin TpFl, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MIC6, MIC7, MIC8), rhoptry protein Rop2, rhoptry proteins (Rop1, Rop2, Rop3, Rop4, Rop5, Rop6, Rop7, Rop16, Rjop17), protein SRI, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IglC1, intracellular growth locus IglC2, aminotransferase Wbtl, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein Dnak, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen; (Ureaplasma urealyticum, Ureaplasma urealyticum infection); non-structural polyprotein, structural polyprotein, capsid protein CP, protein E1, protein E2, protein E3, protease PI, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FlhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase CI, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspA1, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, and protein PsaA (Yersinia enterocolitica, Yersiniosis).

In embodiments wherein the infectious disease is influenza, the messenger RNA molecule may have a coding region encoding at least one antigenic peptide or protein derived from hemagglutinin (HA), neuraminidase (NA), nucleoprotein (NP), matrix protein 1 (MI), matrix protein 2 (M2), non-structural protein 1 (NS1), non-structural protein 2 (NS2), nuclear export protein (NEP), polymerase acidic protein (PA), polymerase basic protein PB1, PB1-F2, or polymerase basic protein 2 (PB2) of an influenza virus or a fragment or variant thereof.

In certain embodiments, the coding region encodes at least one antigenic peptide or protein derived from hemagglutinin (HA) and/or neuraminidase (NA) of an influenza virus or a fragment or variant thereof. The HA and/or NA may, independently, be derived from an influenza A virus or an influenza B virus or a fragment of either.

In embodiments wherein the infectious disease is influenza, the messenger RNA molecule may have a coding region encoding at least one antigenic peptide or protein derived from Spike(S) protein.

In embodiments, the present LNP composition comprises messenger RNA that encodes for an antigen associated with a cancer of a subject or identified from a cancer cell of a subject. The messenger RNA may encode for an antigen determined from a subject's own cancer cell to provide a personalized cancer vaccine.

In embodiments, the present LNP composition comprises messenger RNA that encodes for an antibody. In embodiments, the antibody may be a bi-specific antibody or part of a fusion protein.

### EXAMPLES

### Example 1: Synthesis of Compound 1 (SL01) and Compound 11 (SL02)

Total syntheses of compounds 1 (SL-01) and 11 (SL-02) are shown below. Simple modifications of conditions or the nature of any particular substrate can be changed to achieve the other compounds.

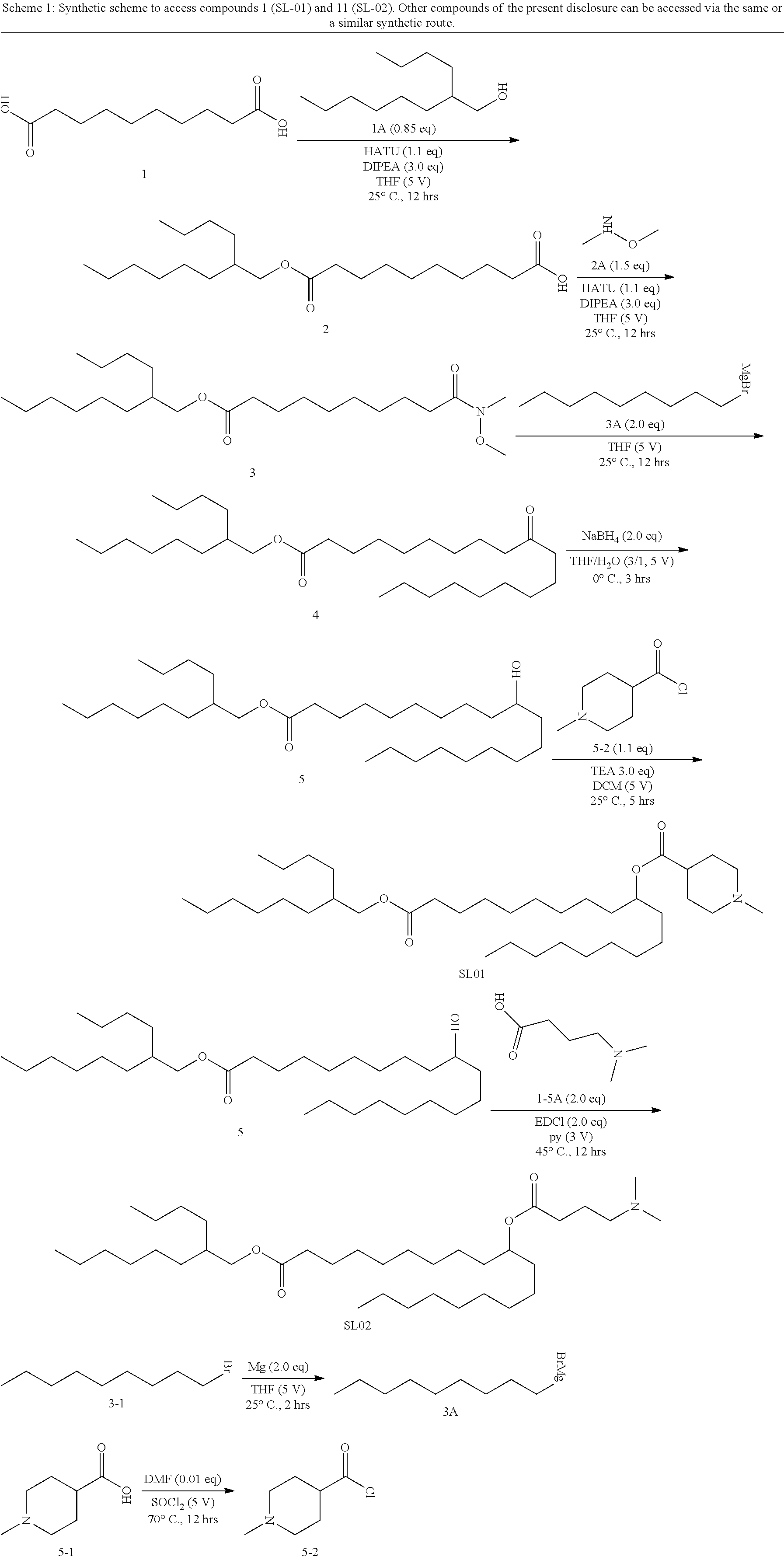

### Synthesis of Compound 11 (SL-02)

### Step 1: Esterification

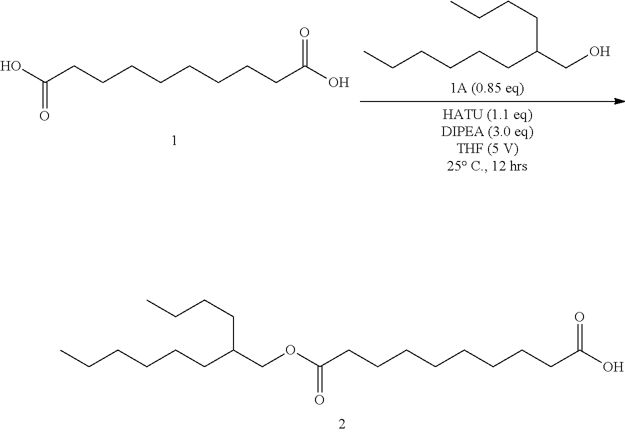

A solution of compound 1 (30.0 g, 148 mmol, 1.00 eq), compound 1A (22.1 g, 118 mmol, 0.85 eq), TEA (45.1 g, 445 mmol, 61.9 mL, 3.00 eq) and HATU (84.6 g, 223 mmol, 1.1 eq) in DCM (150 mL) was stirred at 25° C. for 1 hr. TLC (Petroleum ether/Ethyl acetate=2/1, Rr of product=0.34) showed the starting material was consumed completely. The reaction mixture was poured into aqueous NH₄Cl (200 mL), and the solution was extracted with DCM (100 mL×2). The organic layer was extracted with H₂O (100 mL×2). The organic layer was dried over sodium sulfate and concentrated under reduced pressure to give a residue. The residue was purified by column chromatography (SiO₂, Petroleum ether/Ethyl acetate=500/1 to 0/1) to give compound 2 (41.0 g, 110 mmol, 74.5% yield) as a yellow oil. 1H NMR (400 MHZ, CDCl₃): δ 3.94 (d, J=5.6 Hz, 2H), 2.32-2.25 (m, 4H), 1.60-1.59 (m, 5H), 1.34-1.21 (m, 25H), 0.88-0.84 (m, 6H).

### Step 2: Weinreb Amide Synthesis

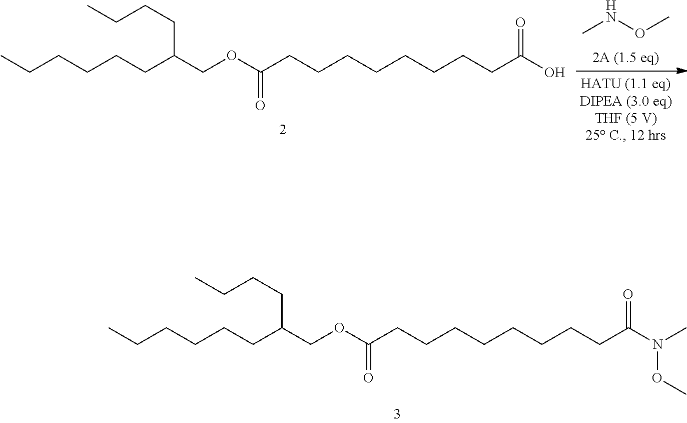

To a solution of compound 2 (15.0 g, 40.5 mmol, 1.00 eq), compound 2A (8.14 g, 60.7 mmol, 1.50 eq, HCl salt) and DIPEA (26.1 g, 202 mmol, 35.2 mL, 3.00 eq) in DMF (80.0 mL) was added HATU (23.1 g, 60.7 mmol, 1.10 eq) at 25° C. After addition, the reaction solution was stirred at 25° C. for 12 hrs. TLC (Petroleum ether/Ethyl acetate=5/1, Rr of product=0.50) showed the reaction finished. The reaction was poured into water (100 mL). The organic phase was collected and the aqueous phase was extracted with ethyl acetate (100 mL×2). The combined organic phase was dried over anhydrous Na₂SO₄ and concentrated under vacuum to give the crude product as oil. The crude product was purified by column chromatography (SiO₂, Petroleum ether/Ethyl acetate=I/O to 0/1) to give compound 3 (12.0 g, 29.1 mmol, 71.7% yield) as a yellow oil. 1H NMR (400 MHZ, CDCl₃): δ 3.96 (d, J=5.6 Hz, 2H), 3.67 (s, 3H), 3.17 (s, 3H), 2.40 (t, J=7.6 Hz, 2H), 2.31-2.27 (m, 2H), 1.69-1.61 (m, 5H), 1.31-1.25 (m, 24H), 0.89-0.80 (m, 6H).

### Step 3: Grignard Reaction

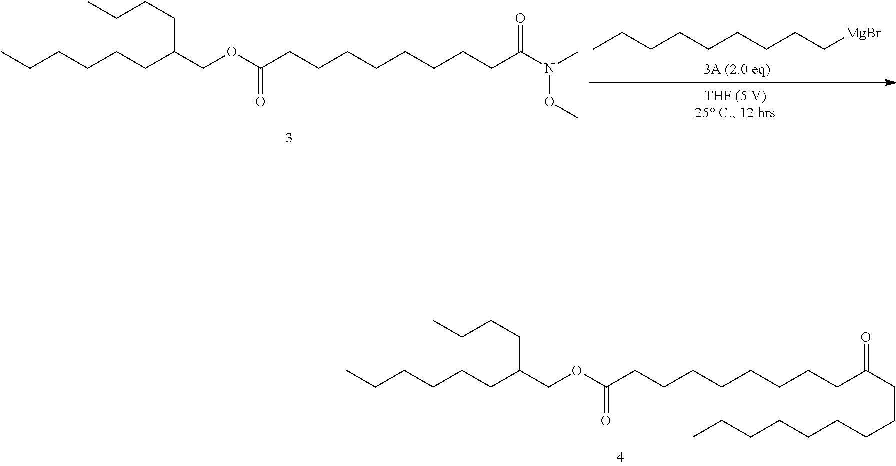

To a solution of compound 3 (7.50 g, 18.3 mmol, 1.00 eq) in THF (20.0 mL) was added compound 3A (1 M, 36.2 mL, 2.00 eq). The reaction was stirred at 25° C. for 5 hrs. TLC (Petroleum ether/Ethyl acetate=5/1, Rr of product=0.78) showed the starting material was consumed completely. The reaction solution poured into the aqueous of NH₄Cl (150 mL), and the suspension was extracted with ethyl acetate (40.0 mL×2). The organic layer was dried over sodium sulfate and concentrated under reduced pressure to give a residue. The residue was purified by column chromatography (SiO₂, Petroleum ether/Ethyl acetate=500/1 to 0/1) to give compound 4 (3.20 g, 6.66 mmol, 36.7% yield) as colorless oil. ¹H NMR (400 MHZ, CDCl₃): δ 3.96 (d, J=5.6 Hz, 2H), 2.37 (t, J=7.6 Hz, 4H), 2.28 (t, J=7.6 Hz, 2H), 1.60-1.53 (m, 7H), 1.27-1.26 (m, 36H), 0.90-0.85 (m, 9H).

### Step 4: Reduction

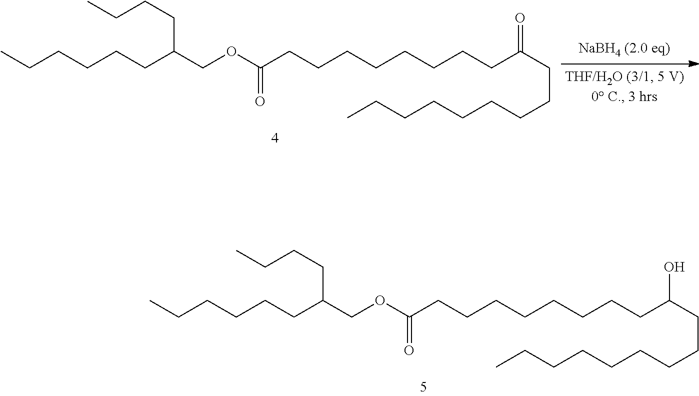 [SMILES: CCCCCCCCCC(=O)CCCCCCCCC(=O)OCC(CCCC)CCCCCC ; CCCCCCCCCC(O)CCCCCCCCC(=O)OCC(CCCC)CCCCCC]

To a solution of compound 4 (2.20 g, 4.58 mmol, 1.00 eq) in THF (10.0 mL) and H₂O (1.00 mL) was added NaBH₄ (346 mg, 9.15 mmol, 2.00 eq). The solution was stirred at 0° C. for 2 hrs. TLC (Petroleum ether/Ethyl acetate=8/1, Rr of material=0.77) showed the starting material was consumed completely. The reaction mixture was poured into the aqueous of NH₄Cl (100.0 mL), and the solution was extracted with ethyl acetate (30.0 mL×2). The organic layer was extracted with H₂O (30.0 mL×2). The organic layer was dried over sodium sulfate and concentrated under reduced pressure to give compound 5 (2.20 g, 4.56 mmol, 99.58% yield) as a colorless oil, which was used to next step directly. 1H NMR (400 MHZ, DMSO-d₆): δ 3.97 (d, J=5.6 Hz, 2H), 3.58 (s, 1H), 2.30 (t, J=7.2 Hz, 2H), 1.62-1.31 (m, 4H), 1.43-1.28 (m, 43H), 0.90-0.87 (m, 9H).

### Step 5: Synthesis of SL-02

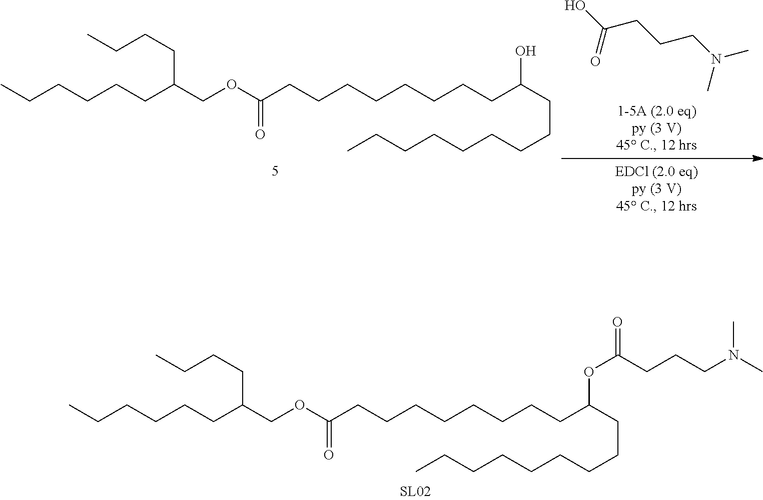

To a solution of compound 5 (2.20 g, 4.56 mmol, 1.00 eq) in Py (8.00 mL) was added compound 1-5A (1.53 g, 9.11 mmol, 2.00 eq, HCl). EDCI (1.75 g, 9.11 mmol, 2.00 eq) was added into the solution. The solution was stirred at 45° C. for 12 hrs. LCMS showed the starting material was consumed completely. The reaction mixture was poured into the aqueous of NH₄Cl (100 mL), and the solution was extracted with ethyl acetate (20.0 mL×2). The organic layer was washed with H₂O (30.0 mL×2). The organic layer was dried over sodium sulfate and concentrated under reduced pressure to give a residue. The residue was purified by column chromatography (SiO₂, Petroleum ether/Ethyl acetate=80/1 to 0/1) to give SL-02 (1.00 g, 1.68 mmol, 36.8% yield) as a light yellow oil. LCMS: (RT=2.291 min, M+H⁺=596.6)

¹H NMR (400 MHZ, CDCl₃): δ 5.79-5.71 (m, 2H), 5.58-5.52 (m, 2H), 4.50 (d, J=6.4 Hz, 4H), 2.38-2.34 (m, 4H), 2.30-2.26 (m, 4H), 2.04-2.03 (m, 4H), 1.60-1.54 (m, 8H), 1.36-1.27 (m, 32H), 0.89-0.85 (m, 6H).

### Synthesis of Grignard Reagent

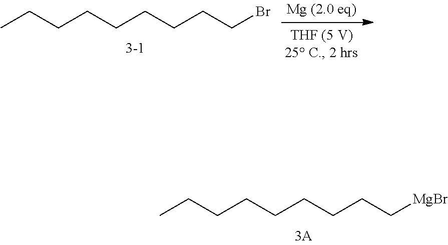

To a solution of Mg (11.7 g, 482 mmol, 10.0 eq) in THF (50.0 mL), a solution of compound 3-1 (10 g, 48.27 mmol, 1.00 eq) in THF (10.0 mL) was added to the solution dropwise. The resulting suspension was stirred at 25° C. for 2 hrs. TLC (Petroleum ether/Ethyl acetate=5/1, Rr of product=0.3) showed the reaction was finished. The reaction solution was used for next step without work-up. The reaction solution was used for the next step directly.

### Synthesis of Compound 1 (SL-01)

Steps 1 to 4 were followed as outlined above.

### Step 5: Synthesis of SL-01

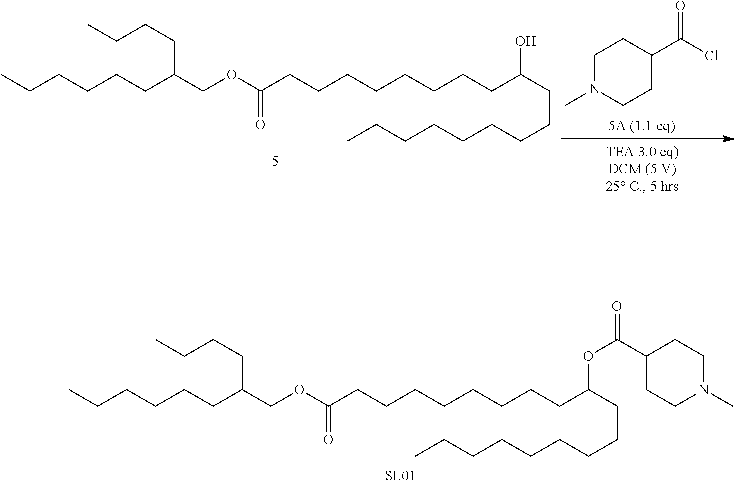

To a solution of compound 5 (4.00 g, 8.28 mmol, 1.00 eq) in DCM (20.0 mL) was added TEA (2.51 g, 24.8 mmol, 3.46 mL, 3.00 eq). Compound 5 (2.01 g, 12.4 mmol, 1.50 eq) was added the solution. The solution was stirred at 20° C. for 4 hrs. TLC (Petroleum ether Ethyl acetate=3/1, R_{f} of product=0.60) showed the reaction was finished. The reaction solution poured into the H₂O (100 mL), and the suspension was extracted with ethyl acetate (50.0 mL×2). The organic layer was dried over sodium sulfate and concentrated under reduced pressure to give a residue. The residue was purified by column chromatography (SiO₂, Petroleum ether/Ethyl acetate=300/1 to 0/1) to give SL-01 (1.00 g, 1.64 mmol, 19.8% yield) as a colorless oil. 1H NMR (400 MHZ, CDCl₃): δ 4.88-4.85 (m, 1H), 3.97 (d, J=5.6 Hz, 2H), 2.82-2.79 (m, 2H), 2.31-2.26 (m, 6H), 2.01-1.95 (m, 2H), 1.91-1.87 (m, 2H), 1.82-1.75 (m, 2H), 1.62-1.57 (m, 3H), 1.50-1.49 (m, 4H), 1.28-1.25 (m, 40H), 0.90-0.86 (m, 9H).

### Synthesis of Acid Chloride Building Block for SL-01

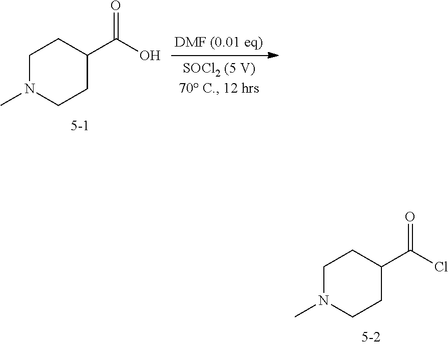

To a solution of compound 5-1 (5.00 g, 34.9 mmol, 1.00 eq) in SOCl₂ (25.0 mL) was added DMF (25.5 mg, 349 umol, 26.8 uL, 0.01 eq), the reaction solution was stirred at 70° C. for 12 hrs. A sample was taken to be quenched by MeOH. TLC (Petroleum ether Ethyl acetate=5/1, Rr of product=0.71) showed the starting material was consumed completely. The reaction solution was concentrated under vacuum to give a residue. The residue was dissolved in Tol. (20.0 mL×3) and concentrated under vacuum twice to give compound 5-2 (5.60 g, 34.6 mmol, 99.2% yield) as a light yellow oil, which was used to next step directly.

Other compounds of the present invention can be synthesised using the same or a similar synthetic protocol as outlined above.

### Example 2: Formulation of Compound into LNPs with a Messenger RNA

Messenger RNA-containing lipid nanoparticle compositions were prepared using ionizable cationic lipids selected from Compounds 1-12, specifically Compound 1 (SL01) and Compound 11 (SL02), additional helper lipids, and self-amplifying mRNA (saRNA) encoding proteins of interest. In general terms, the messenger RNA-LNP was formulated by the controlled mixing of an ethanol solution containing the lipid components and an aqueous solution containing citrate buffer at pH 6 containing the saRNA. The resulting LNP was then buffer exchanged to a neutral pH Tris cryobuffer containing sucrose by using either tangential flow filtration (TFF) or centrifugal ultrafiltration (UF).

Formulation of H5-Luciferase expressing saRNA into Compound 1 LNPs. Compound 1, 1,2-distearoyl-sn-glycero-3-phosphocholine (DSPC), cholesterol, and 1,2-dimyristoyl-rac-glycero-3-methylpolyoxyethylene (DMG-PEG) were combined in a 40:10:48:2 molar ratio in ethanol at a concentration of 3.2 mM. A solution of saRNA expressing H5-Luciferase in 50 mM citrate buffer at pH 6 with 10 mM Tris(2-carboxyethyl) phosphine (TCEP) was prepared at 0.025 mg/mL. The lipid solution in ethanol was then rapidly mixed with the saRNA solution using a staggered herringbone micromixer chip using Nanoassemblr benchtop instrument (Precision Nanosystems) at a total flow rate (TFR) of 12 mL/min and a flow rate ratio (FRR) of 2:1 aqueous buffer to ethanol. This mixing ratio results in a 8:1 molar ratio of ionizable cationic lipid (Compound 1) to saRNA phosphate groups and a total lipid to saRNA mass ratio of 37:1. The resulting mixed solution was then diluted 10-fold into 50 mM citrate buffer at pH 6 with 10 mM TCEP and subjected to tangential flow filtration (TFF) using a 300 k molecular weight cut-off membrane (mPES) until concentrated to the original volume. Subsequently, the citrate buffer was replaced with a buffer containing 20 mM Tris buffer at pH 7.5, 80 mM sodium chloride, and 3% sucrose using diafiltration with 10 diavolumes. The LNP solution was concentrated to volume of 6.2 mL, filtered using a 0.2 micron PES syringe filter, aliquoted into vials, and frozen at 1° C./min using a Corning® CoolCell® LX Cell Freezing Container until the samples reached −80° C. Samples were stored at −80° C. and thawed on wet ice before analysis or use. Based on a ribogreen assay, the total RNA concentration was 41.6 μg/mL, 49.1% of the input RNA was recovered, and the encapsulation efficiency was 85.9%. As measured by dynamic light scattering (DLS), the LNPs had a Z-avg diameter of 116.1 nm and a polydispersity index (PDI) of 0.15 (see Table 1, entry 4).

Formulation of H5N1 expressing saRNA into Compound 1 LNPs. Compound 1, 1,2-distearoyl-sn-glycero-3-phosphocholine (DSPC), cholesterol, and 1,2-dimyristoyl-rac-glycero-3-methylpolyoxyethylene (DMG-PEG) were combined in a 40:10:48:2 molar ratio in ethanol at a concentration of 3.2 mM. A solution of saRNA expressing H5-N1 influenza antigens in 50 mM citrate buffer at pH 6 with 10 mM Tris(2-carboxyethyl) phosphine (TCEP) was prepared at 0.025 mg/mL. The lipid solution in ethanol was then rapidly mixed with the saRNA solution using a staggered herringbone micromixer chip using Nanoassemblr benchtop instrument (Precision Nanosystems) at a total flow rate (TFR) of 12 mL/min and a flow rate ratio (FRR) of 2:1 aqueous buffer to ethanol. This mixing ratio results in a 8:1 molar ratio of ionizable cationic lipid (Compound 1) to saRNA phosphate groups and a total lipid to saRNA mass ratio of 37:1. The resulting mixed solution was then diluted 10-fold into 50 mM citrate buffer at pH 6 with 10 mM TCEP and subjected to tangential flow filtration (TFF) using a 300 k molecular weight cut-off membrane (mPES) until concentrated to the original volume. Subsequently, the citrate buffer was replaced with a buffer containing 20 mM Tris buffer at pH 7.5, 80 mM sodium chloride, and 3% sucrose using diafiltration with 10 diavolumes. The LNP solution was concentrated to volume of 6.5 mL, filtered using a 0.2 micron PES syringe filter, aliquoted into vials, and frozen at 1° C./min using a Corning® CoolCell® LX Cell Freezing Container until the samples reached −80° C. Samples were stored at −80° C. and thawed on wet ice before analysis or use. Based on a ribogreen assay, the total RNA concentration was 51.3 μg/mL, 31.8% of the input RNA was recovered, and the encapsulation efficiency was 89.3%. As measured by dynamic light scattering (DLS), the LNPs had a Z-avg diameter of 122.5 nm and a polydispersity index (PDI) of 0.03 (see Table 1, entry 1).

Formulation of H5-Luciferase expressing saRNA into Compound 11 LNPs. Compound 11, 1,2-distearoyl-sn-glycero-3-phosphocholine (DSPC), cholesterol, and 1,2-dimyristoyl-rac-glycero-3-methylpolyoxyethylene (DMG-PEG) were combined in a 40:10:48:2 molar ratio in ethanol at a concentration of 3.2 mM. A solution of saRNA expressing H₅-Luciferase in 50 mM citrate buffer at pH 6 with 10 mM Tris(2-carboxyethyl) phosphine (TCEP) was prepared at 0.025 mg/mL. The lipid solution in ethanol was then rapidly mixed with the saRNA solution using a staggered herringbone micromixer chip using Nanoassemblr benchtop instrument (Precision Nanosystems) at a total flow rate (TFR) of 12 mL/min and a flow rate ratio (FRR) of 2:1 aqueous buffer to ethanol. This mixing ratio results in a 8:1 molar ratio of ionizable cationic lipid (Compound 11) to saRNA phosphate groups and a total lipid to saRNA mass ratio of 37:1. The resulting mixed solution was then diluted 10-fold into 50 mM citrate buffer at pH 6 with 10 mM TCEP and subjected to tangential flow filtration (TFF) using a 300 k molecular weight cut-off membrane (mPES) until concentrated to the original volume. Subsequently, the citrate buffer was replaced with a buffer containing 20 mM Tris buffer at pH 7.5, 80 mM sodium chloride, and 3% sucrose using diafiltration with 10 diavolumes. The LNP solution was concentrated to volume of 6.2 mL, filtered using a 0.2 micron PES syringe filter, aliquoted into vials, and frozen at 1° C./min using a Corning® CoolCell® LX Cell Freezing Container until the samples reached −80° C. Samples were stored at −80° C. and thawed on wet ice before analysis or use. Based on a ribogreen assay, the total RNA concentration was 43.1 μg/mL, 46.0% of the input RNA was recovered, and the encapsulation efficiency was 89.7%. As measured by dynamic light scattering (DLS), the LNPs had a Z-avg diameter of 108.6 nm and a polydispersity index (PDI) of 0.14 (see Table 1, entry 6).

Formulation of H5N1 expressing saRNA into Compound 11 LNPs. Compound 11, 1,2-distearoyl-sn-glycero-3-phosphocholine (DSPC), cholesterol, and 1,2-dimyristoyl-rac-glycero-3-methylpolyoxyethylene (DMG-PEG) were combined in a 40:10:48:2 molar ratio in ethanol at a concentration of 3.2 mM. A solution of saRNA expressing H5-N1 influenza antigens in 50 mM citrate buffer at pH 6 with 10 mM Tris(2-carboxyethyl) phosphine (TCEP) was prepared at 0.025 mg/mL. The lipid solution in ethanol was then rapidly mixed with the saRNA solution using a staggered herringbone micromixer chip using Nanoassemblr benchtop instrument (Precision Nanosystems) at a total flow rate (TFR) of 12 mL/min and a flow rate ratio (FRR) of 3:1 aqueous buffer to ethanol. This mixing ratio results in a 8:1 molar ratio of ionizable cationic lipid (Compound 1) to saRNA phosphate groups and a total lipid to saRNA mass ratio of 37:1. The resulting mixed solution was then diluted 10-fold into 50 mM citrate buffer at pH 6 with 10 mM TCEP and subjected to tangential flow filtration (TFF) using a 300 k molecular weight cut-off membrane (mPES) until concentrated to the original volume. Subsequently, the citrate buffer was replaced with a buffer containing 20 mM Tris buffer at pH 7.5, 80 mM sodium chloride, and 3% sucrose using diafiltration with 10 diavolumes. The LNP solution was concentrated to a volume of 4.8 mL, filtered using a 0.2 micron PES syringe filter, aliquoted into vials, and frozen at 1° C./min using a Corning® CoolCell® LX Cell Freezing Container until the samples reached −80° C. Samples were stored at −80° C. and thawed on wet ice before analysis or use. Based on a ribogreen assay, the total RNA concentration was 58.3 μg/mL, 53.3% of the input RNA was recovered, and the encapsulation efficiency was 88.8%. As measured by dynamic light scattering (DLS), the LNPs had a Z-avg diameter of 138.8 nm and a polydispersity index (PDI) of 0.07 (see Table 1, entry 7).

TABLE 1
Example LNP formulations with SL01 and SL02. (Citrate buffer pH was pH 6 in all 9 formulations)

| | | | | Flow-rate | | | | | |
|---|---|---|---|---|---|---|---|---|---|
| | | | Input | ratio | | | | Z-average | |
| | Ionizable | Antigens | RNA | (FRR, | Encapsulation | Total RNA | RNA | diameter | Polydispersity |
| LNP | Cationic | expressed | scale | Aqueous: | efficiency | concentration | recovery | by DLS | index (PDI) by |
| # | Lipid | by saRNA | (mg) | Ethanol) | (EE, %) | (μg/mL) | (%) | (nm) | DLS |
| 1 | SL01 | H5, N1 | 0.5 | 2:1 | 89.3 | 51.3 | 31.8 | 122.5 | 0.03 |
| 2 | SL01 | H5, | 1.5 | 2:1 | 90.3 | 142.5 | 41.6 | 110.0 | 0.11 |
| | | Luciferase | | | | | | | |
| 3 | SL01 | H5, N1 | 1.5 | 2:1 | 89.5 | 138.5 | 52.7 | 118.7 | 0.11 |
| 4 | SL01 | H5, N1 | 0.5 | 2:1 | 85.9 | 41.6 | 49.1 | 116.1 | 0.15 |
| 5 | SL02 | H5, N1 | 0.5 | 2:1 | 83.3 | 59.0 | 23.6 | 194.2 | 0.04 |
| 6 | SL02 | H5, N1 | 0.5 | 2:1 | 89.7 | 43.1 | 46.0 | 108.6 | 0.14 |
| 7 | SL02 | H5, N1 | 0.5 | 3:1 | 88.8 | 58.3 | 53.3 | 138.8 | 0.07 |
| 8 | SL02 | H5, N1 | 1.5 | 3:1 | 91.9 | 153.6 | 54.6 | 86.3 | 0.10 |
| 9 | SL02 | H5, N1 | 1.5 | 3:1 | 94.3 | 128.9 | 54.0 | 117 | 0.03 |

### Biophysical Characterization of Lipid Nanoparticles

The messenger RNA-containing LNP composition was characterized using analytical methods to determine the loading of messenger RNA, the percentage of messenger RNA that is encapsulated, and the size of the particles. The total amount of messenger RNA contained in the sample and the percentage of that messenger RNA that is encapsulated was determined using a fluorescence assay employing Ribogreen, a dye that becomes more emissive upon binding messenger RNA. The total amount of messenger RNA was determined by disrupting the LNP with 1 wt % Triton-X 100 to expose the encapsulated messenger RNA, adding the dye, and comparing the emission intensity against a standard curve prepared using ribosomal RNA. The amount of unencapsulated messenger RNA was measured in a similar manner with the detergent disruption of the LNP is omitted. With the total amount of messenger RNA known and the amount of unencapsulated messenger RNA known, the percent encapsulated messenger RNA was calculated thus:

$$Percent Encapsulation ( \% ) = ( ( {RNA}_{TOTAL} - {RNA}_{UNENCAPSULATED} ) / {RNA}_{TOTAL} ) \times 100$$

where RNA_{TOTAL} and RNA_{UNENCAPSULATED} are, respectively, the concentrations of total messenger RNA and unencapsulated messenger RNA. The total messenger RNA content varied based on formulation, but generally fell in the range of 0.030-0.200 mg/mL. The size of LNP was measured using dynamic light scattering of a sample diluted 1:100 in PBS buffer.

### pKa Protocol

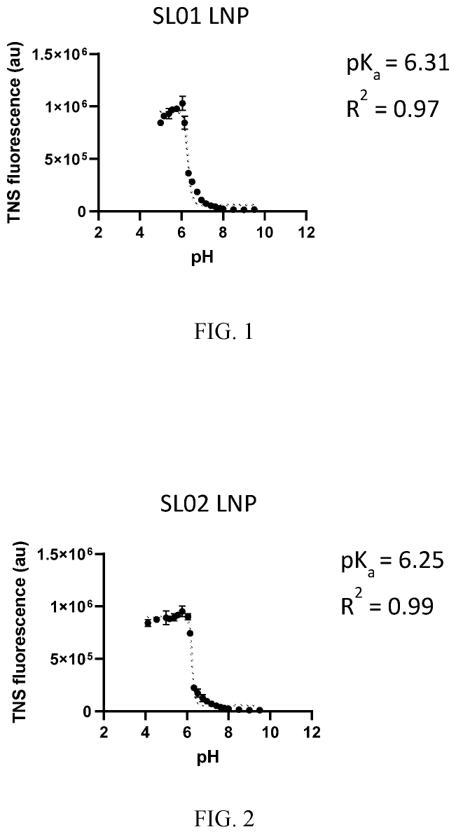

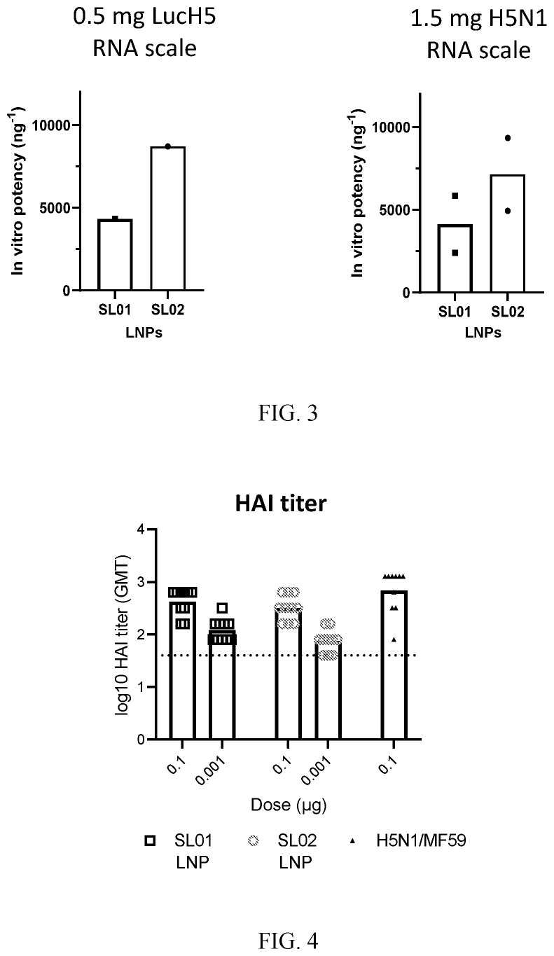

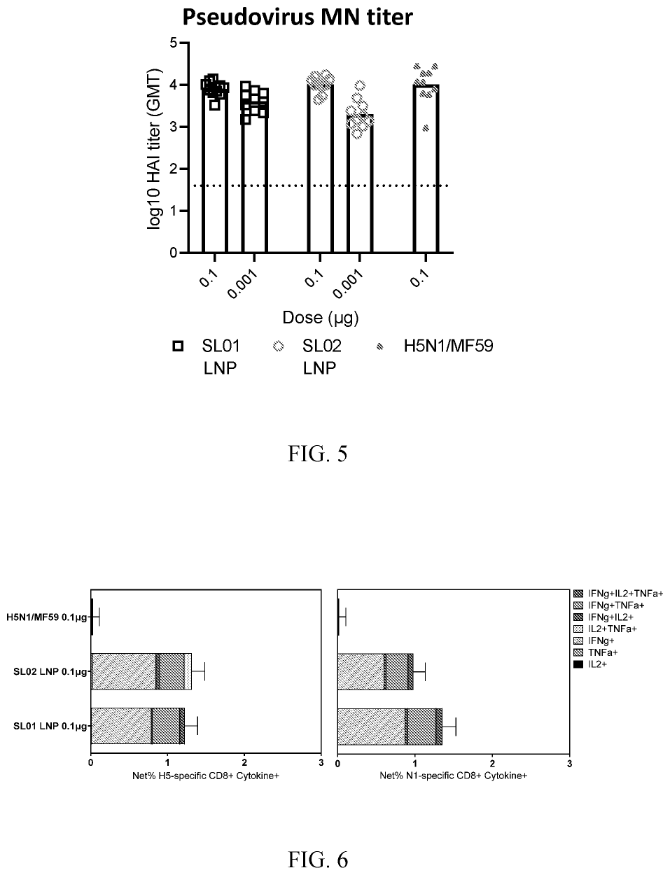

In a black 96-well plate, solutions of saRNA-LNP (final assay concentration 2 μg/mL total RNA) in a series of buffers ranging from pH 4 to 9.5 were prepared. Buffers between pH4 to 7.6 were prepared from disodium phosphate and citric acid. Buffers from 7.8 to 9.5 were prepared by titrating tris buffer with 10 N sodium hydroxide. To each well was added 6-(p-Toluidino)-2-naphthalenesulfonic acid sodium salt (TNS) in water to a final assay concentration of 6 μM. The fluorescence was read on a plate reader at 25° C. with an excitation setting of 321 nm and an emission setting of 445 nm. The intensity values were plotted as a function of pH using GraphPad Prism and were fit with a sigmoidal dose response curve. The apparent pKa was determined as the EC50 of this curve where half of the ionizable amines are expected to be protonated. The apparent pKa values of saRNA-LNPs with SL01 and SL02 were 6.31 and 6.25, respectively (FIGS. 1 and 2).

### Characterization of the In Vitro Potency of LNPs

The ability of the saRNA-LNPs to transfect cultured cells was characterized using an in vitro assay based on the percentage of cells expressing the antigen of interest. Specifically, 1 million BHK-V cells were co-incubated with saRNA-LNP of varying concentration (highest 50 ng per well, lowest 0.39 ng per well) in 0.3 mL of media for 17-19 hours at 37° C. with 5% CO₂. Subsequently, the cells were scraped from the dish to form a single-cell suspension, fixed and permeabilized (BD Cytofix/Perm kit), and then stained with fluorophore-labeled antigen-specific antibodies against H5 and N1 (for saRNA expressing both H5 and N1). Luciferase expression was not measured for saRNA expressing both H5 and luciferase. The percentage of single or double antigen positive cells was quantified using a BD Accuri flow cytometer. The natural log of the fraction of cells that did not express antigen was plotted against the mass (ng) of RNA added and linear portion was fit to a straight line using a linear regression. The slope of that line multiplied by −100,000 was reported as the LNP potency. Alternatively, a similar assay was performed in a 96-well using BHK-21 cells and analysed using a Cytation 5 Cell Imaging Multimode Reader (Agilent). The results of this assay were analysed in the same way as described for the flow cytometry assay. The data shows that both SL01 and SL02 are able to transfect BHK-V cells in vitro. SL02 LNPs trended higher in potency for both saRNA constructs examined (FIG. 3).

### Characterization of the In Vivo Potency of Lipid Nanoparticles

The ability of an saRNA LNP to act as a vaccine was evaluated using a in vivo mouse immunogenicity study. Groups of 10 6-8 week old female Balb/c mice were administered either 0.1 μg saRNA LNP or 0.001 μg saRNA as an intramuscular injection on study day 0 and and on study day 21. On study day 42 the mice were euthanised and serum and spleens were collected for serological and cell-based immune assays. The vaccine antibody response in serum was measured using an IgG ELISA, a hemagglutination inhibition assay (HAI), a pseudovirus microneutralization assay (MN), and an enzyme linked lectin assay (ELLA). As shown in FIGS. 4 and 5, LNPs encapsulating saRNA expressing influenza antigens H5 and N1 led to high levels of neutralizing antibodies in mice as measured by a hemagglutination inhibition (HAI) assay and a pseudovirus microneutralization (MN) assay following a prime-boost vaccination schedule. At a 0.1 μg saRNA dose, the values for both LNPs were comparable to those elicited by an inactivated virus vaccine adjuvanted with MF59.

Antigen-specific T cells (both CD4+ and CD8+) were evaluated using intracellular cytokine staining following peptide-stimulation. As shown in FIG. 6, SL01 and SL02 LNPs encapsulating saRNA expressing influenza antigens H5 and N1 led to measurable levels of activated antigen-specific CD8 T cells following a prime-boost vaccination schedule in contrast to an inactivated virus vaccine adjuvanted with MF59.

### Characterization of the In Vivo Transfection Efficiency of LNPs

The efficiency and location of in vivo translation of saRNA into protein when delivered with an LNP will also be evaluated using Luciferase-H5 saRNA LNPs based on whole mouse bioluminescence imaging. For example, 1.0 μg or 0.1 μg saRNA LNP will be injected intramuscularly in the hind leg. At defined time points such as daily, the mice will be administered luciferin and the bioluminescence will be imaged and quantified.

### Characterization of Genotoxic Potential, Tolerability, Biodistribution, and Biodegradability

A standard test battery for prediction of genotoxic potential (damage of DNA) will be used, as no single test can detect all genotoxic mechanisms leading to tumorigenicity. As an example, when a positive result is seen in in vitro mammalian cell assay, clearly negative results in two in vivo assays, in appropriate tissues and demonstrated sufficient test substance exposure, will be considered evidence for lack of genotoxic potential in vivo. Relevant guidelines, such as S2 (R1) Genotoxicity Testing and Data Interpretation for Pharmaceuticals Intended for Human Use”, will be followed.
Test for gene mutation in bacteria.
In vitro cytogenetic test for chromosomal damage (metaphase chromosome aberration or micronucleus test) or mouse lymphoma Tk gene mutation assay.
In vivo genotoxicity test for chromosomal damage using rodent hematopoietic cells either for micronuclei or chromosomal aberrations in metaphase cells.

### In Silico Screening of Novel Lipids for Toxicity

Novel ionizable cationic lipids will be screened for potential toxicity and mutagenicity using commercially available computational toxicology assessment products and/or services to satisfy International “Council for Harmonisation of Technical Requirements for Pharmaceuticals for Human Use (ICH) M7 (R1)_Assessment and Control of DNA Reactive (Mutagenic) Impurities in Pharmaceuticals To Limit Potential Carcinogenic Risk”. For example, two complementing Quantitative (Q) SAR methods, expert rule-based Derek Nexus and statistical-based Sarah Nexus (or Leadscope), will be used to predict outcome of bacterial mutagenicity. Classification will be based on Class 1 to 5 with respect to mutagenic and carcinogenic potential from known mutagenic carcinogen (Class 1) to lack of mutagenicity or carcinogenicity (Class 5). The outcome of computer-based analysis will be reviewed for relevance of positive, negative, conflicting, or inconclusive prediction and rationale for conclusion provided.

As most structural alerts are based on bacterial mutagenicity, compounds with structural alert can be detected in standard test battery. In addition, some chemical classes are more easily detected in mammalian cell chromosome damage assays than bacterial mutation assays. Negative result from compound with structural alert in either test battery should be considered not genotoxic.

### In Vitro Screening for Genotoxicity

Novel lipids would be assessed first for mutagenicity in bacterial reverse gene mutation test (Ames), which detects relevant genetic changes and most genotoxic rodent and human carcinogens and then for genotoxicity in vitro micronucleus (MN) assay using mammalian cells:

Ames assay would follow Organization for Economic Co-operation and Development (OECD) Guidelines for the Testing of Chemicals No. 471 Bacterial Reverse Mutation Test and S2 (R1) Genotoxicity Testing and Data Interpretation for Pharmaceuticals Intended for Human Use to assess mutagenic potential. At least five strains of bacteria Salmonella typhimurium TA98, TA100, TA1537, TA1535 and TA102 or E. coli WP2 will be exposed to test substance in the presence and absence of metabolic activation system rat liver metabolizing system (S-9).

In vitro mammalian cell micronucleus genotoxicity test would follow OECD Guidelines for the Testing of Chemicals No. 487. This test should detect MN in the cytoplasm of interphase cells and chromosome damaging potential (aneugens and clastrogens). Sufficiently validated and appropriate are mouse lymphoma L5178Y cell Tk (thymidine kinase) gene mutation assay (MLA) and human lymphocyte micronucleus assay (HLM) treated in the absence and presence of rat liver metabolising system (S-9).

### In Vivo Screening for Genotoxicity

Novel lipids would be assessed in vivo in either acute or repeat-dose rat study, as some agents are mutagenic in vivo but not in vitro. The choice of analysis will be micronuclei in erythrocytes (in blood or bone marrow) or chromosome aberrations in metaphase cells in bone marrow.

### Biodistribution and Biodegradability Assessment

Ex vivo biodegradability assessment: Novel lipids will be screened for enzymatic biodegradation using enzyme-containing solutions prepared from relevant species, such as human, mouse, and rat. Lipids will be screened both in their neat form (unformulated) and when incorporated into LNPs that do or do not contain RNA. For example, a novel ionizable cationic lipid will be diluted at an approximate concentration of 1.0 to 0.001 mg/mL in an aqueous solution of human or rat liver microsomes and incubated at temperatures in the range 25-37° C. for durations from 0.1-24 hours. Subsequently, the amount of intact lipid remaining will be measured using liquid chromatography (LC) with evaporative light scattering (ELS) or mass spectrometry (MS) detection and compared to control samples that were not treated with enzymes or were incubated at 2-8° C. to inhibit enzymatic activity. Furthermore, the appearance of new peaks in the chromatograph, which presumably represent degradation products, will be investigated using MS to confirm identity. Without wishing to be bound by theory, the expectation is that degradation may occur via ester hydrolysis and that the lipid's structure will influence the rate of ester hydrolysis.

In vivo biodistribution, pharmacokinetics, and biodegradability assessment: Assessment of biodistribution and pharmacokinetics [Absorption, Distribution, Metabolism, and Excretion) (ADME))] will be performed in rat, which will receive the novel lipids (in their neat form and/or incorporated into LNPs that do or do not contain RNA) intramuscularly or intravenously.

Assessment of biodistribution will be based on the principles of ICH-M3 (R2) and World Health Organization (WHO) guidelines on nonclinical evaluation of vaccines, WHO Technical Report Series No. 927, Annex 1. Blood samples will be collected multiple times for lipid, mRNA and immunogenicity analysis. Tissues will be collected at predefined days and lipid analyses performed with qualified liquid chromatography-mass spectrometry method (LS-MS/MS). If needed, RT qPCR analyses of the nucleic acid payload will be performed.

Lipid pharmacokinetics in plasma will be evaluated with qualified LS-MS/MS.

### In Vivo Tolerability Assessment

The tolerability of novel lipids will be evaluated in appropriate species, such as mice or rats. For example, groups of Sprague-Dawley rats will be administered novel-lipid-containing RNA LNP vaccines at doses ranging from 0.1 to 30 μg total RNA per animal via intramuscular injection. Each animal will be given between 1-3 injections and the effect of treatment on weight loss, food intake, and body temperature will be measured. In addition, the injection site will be monitored using the Draize dermal irritation scoring system. In addition, serum will be collected to evaluate the vaccine immune response using ELISA and functional antibody assays. In addition, the complete blood count, blood chemistry, and blood coagulation will be assessed at one or more timepoint during the study. At the end of the study during necropsy select organs may be macroscopically assessed and weighed. Novel lipids associated with RNA LNP vaccines that maintain high immunogenicity while causing the least undesired responses, such as weight loss, fever, or injection site reactogenicity, will be considered more promising candidates over lipids with a less attractive tolerability profile.

### Toxicity Assessment

Repeat-dose general toxicity study: Toxicity of the candidate novel lipids will be assessed in a pharmacological relevant nonclinical species (e.g. rat). Novel-lipid-containing RNA/LNP vaccines will be administered in Sprague-Dawley rats once every three weeks (total of 3 doses) via intramuscular injection. The reversibility or persistence of any effects will be assessed after a 3-week recovery phase.

Assessment of toxicity will be based on mortality, clinical observations, body weights, food consumption, ophthalmic observations, dose site (dermal) observations, body temperatures, and clinical and anatomic pathology, micronucleus analysis. Blood samples will be collected for immunogenicity analysis.

Novel lipid-containing RNA/LNP vaccines that maintain high immunogenicity with improved reactogenicity/tolerability profiles will be considered for further development.

### Itemized Listing of Embodiments

1. A lipid nanoparticle comprising a messenger RNA and an ionizable cationic lipid compound selected from the group consisting of:

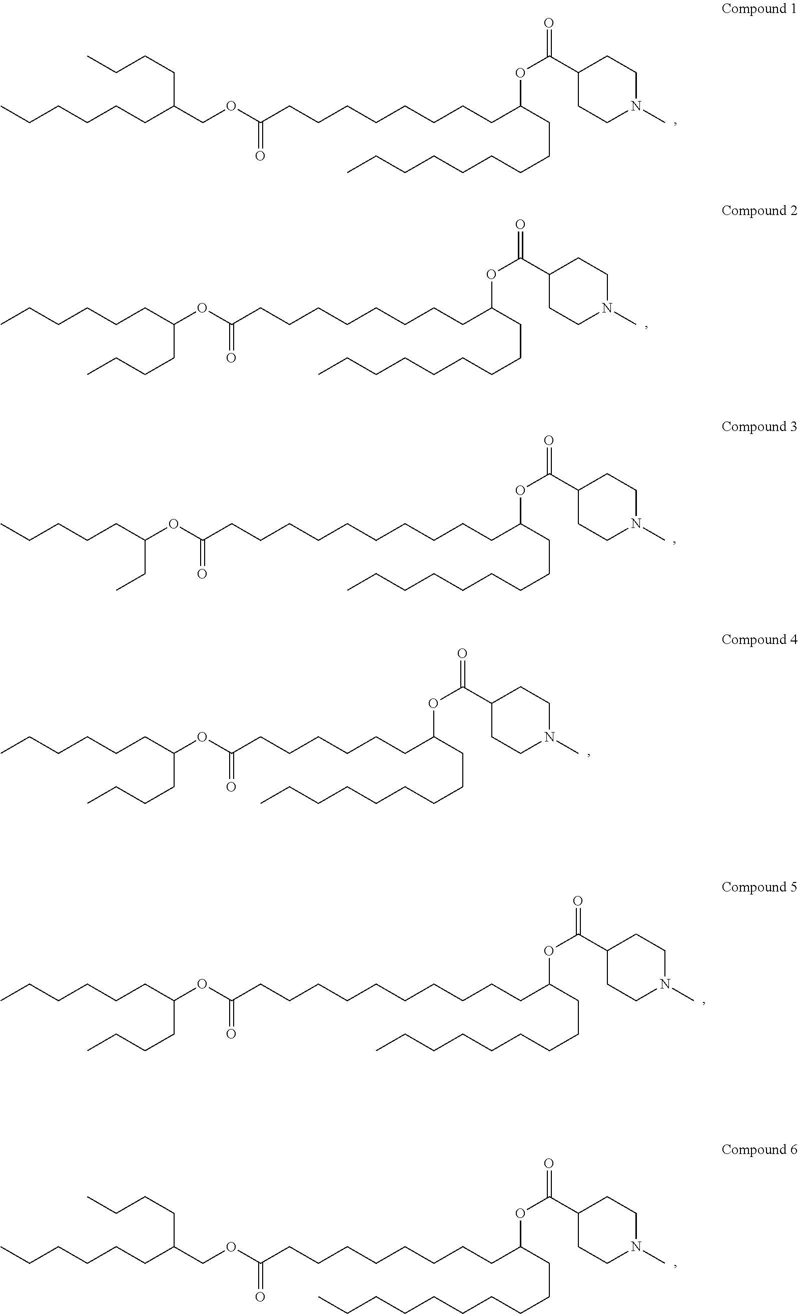

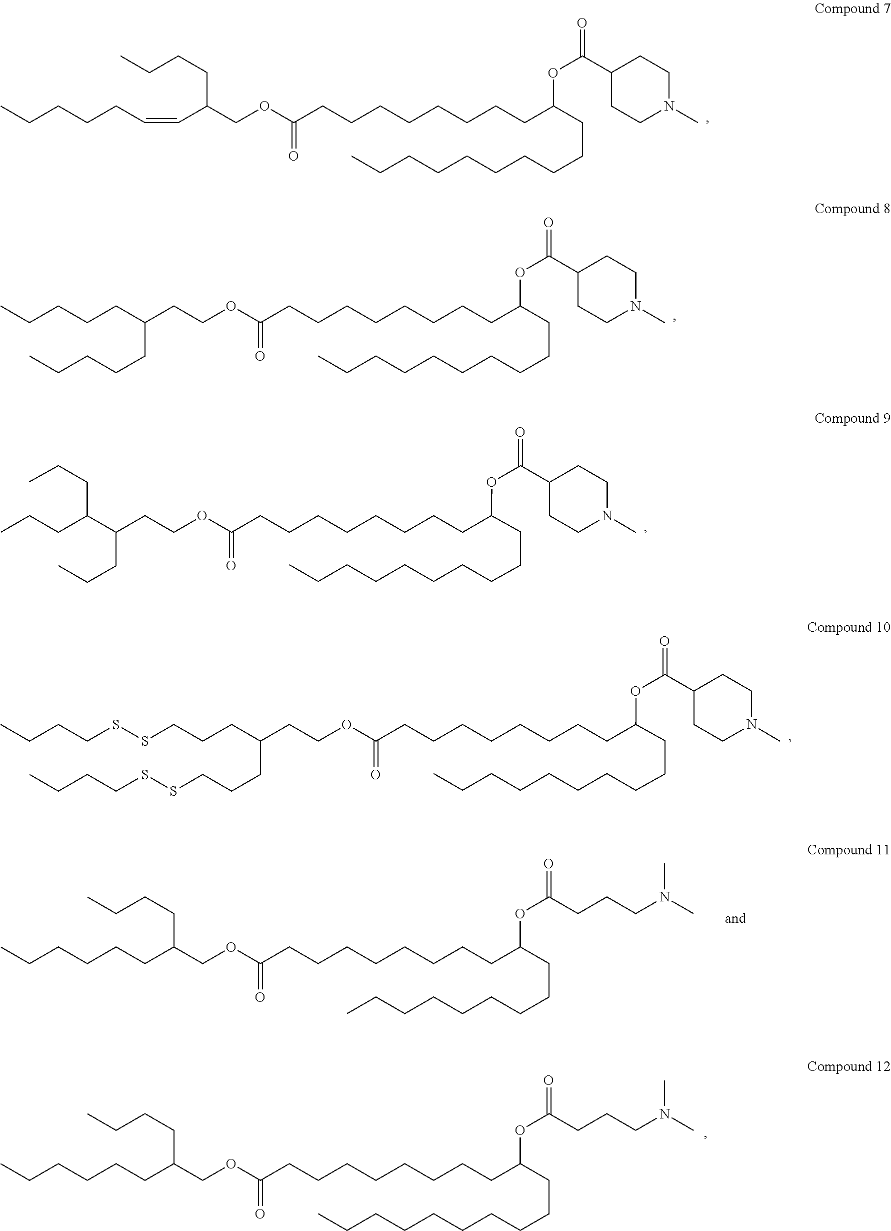

or a pharmaceutically acceptable salt or prodrug thereof.
2. The lipid nanoparticle of item 1, wherein the lipid component further comprises one or more of a neutral lipid, a structural lipid and a PEGylated lipid.
3. The lipid nanoparticle of item 2, wherein the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.
4. The lipid nanoparticle of item 2 or item 3, wherein the structural lipid is selected from the group consisting of cholesterol, fecosterol, sitosterol, campesterol, stigmasterol, brassicasterol, ergosterol, tomatidine, tomatine, ursolic acid and alpha-tocopherol.
5. The lipid nanoparticle of any one of item 2 to item 4, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.
6. The lipid nanoparticle of any one of item 1 to item 5, comprising: about 25 mol % to about 60 mol % of a compound selected from Compounds 1 to 12; about 2 mol % to about 25 mol % neutral lipid; about 18.5 mol % to about 60 mol % structural lipid; and about 0.2 mol % to about 10 mol % of PEGylated lipid.
7 The lipid nanoparticle of any one of item 1 to item 6, wherein the messenger RNA is selected from a conventional messenger RNA and a self-amplifying messenger RNA.
8 The lipid nanoparticle of any one of item 1 to item 7, wherein the conventional messenger RNA and/or self-amplifying messenger RNA comprise a sequence encoding an antigenic peptide or protein, or a fragment, variant or derivative thereof.
9. The lipid nanoparticle of item 8, wherein the antigenic peptide or protein is selected from the group consisting of pathogenic antigens, tumour antigens, allergenic antigens or autoimmune self-antigens.
10. The lipid nanoparticle of item 9, wherein the pathogenic antigens are derived from a bacterial, viral or protozoological pathogenic organisms.
11. The lipid nanoparticle of any one of item 1 to item 10, wherein the lipid nanoparticle has a diameter of from about 30 nm to about 160 nm.
12. The lipid nanoparticle of any one of item 1 to item 11, wherein the lipid nanoparticle comprises a compound selected from Compounds 1-10 and 12.
13. The lipid nanoparticle of any one of item 1 to item 12, wherein the lipid nanoparticle comprises a compound selected from Compounds 1-10.
14. A lipid nanoparticle composition comprising a plurality of lipid nanoparticles of any one of item 1 to item 13, and a pharmaceutically acceptable carrier, excipient or diluent.
15. A method of delivering a messenger RNA to a mammalian cell, including administering the lipid nanoparticle of any one of item 1 to item 13 or the lipid nanoparticle composition of item 14, to a subject to thereby contact the cell with the lipid nanoparticle or lipid nanoparticle composition and deliver the messenger RNA to the cell
16. The method of item 15, wherein the cell is a cell of a human subject.
17. A method of producing a polypeptide of interest in a mammalian cell, including the step of contacting the cell with a lipid nanoparticle of any one of item 1 to item 13, or the lipid nanoparticle composition of item 14, wherein the lipid nanoparticle comprises a conventional messenger RNA or a self-amplifying messenger RNA encoding the polypeptide.
18. A method of treating a disease, disorder or condition in a subject in need of such treatment, comprising administering a lipid nanoparticle of any one of item 1 to item 13, or the lipid nanoparticle composition of item 14, to the subject to thereby treat the disease, disorder or condition.
19. Use of a lipid nanoparticle of any one of item 1 to item 13, or the lipid nanoparticle composition of item 14, in the manufacture of a medicament for the treatment of a disease, disorder or condition.
20. A lipid nanoparticle of any one of item 1 to item 13, or the lipid nanoparticle composition of item 14, for use in the treatment of a disease, disorder or condition in a subject in need of such treatment.
21. The method of item 18, the use of item 19 or the lipid nanoparticle for use of item
20, wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.
22. A vaccine comprising a lipid nanoparticle of any one of item 1 to item 13, or the lipid nanoparticle composition of item 14.
23. The vaccine of item 22, wherein the vaccine is selected from a tumor vaccine, an influenza vaccine, and a SARS, including a SARS-CoV-2, vaccine.
24. The lipid nanoparticle of any one of item 1 to item 13; the lipid nanoparticle composition of item 14; the method of delivering a messenger RNA of item 15 or item 16; the method of producing a polypeptide of interest of item 17; the method of treating a disease, disorder or condition of item 19; the use of a lipid nanoparticle of item 19 or item 21; the lipid nanoparticle for use of item 20 or item 21; the vaccine of item 22 or item 23; wherein the relevant compound is selected from one or more of compound 2 to compound 12.

1. A lipid nanoparticle comprising a messenger RNA and an ionizable cationic lipid compound selected from the group consisting of: 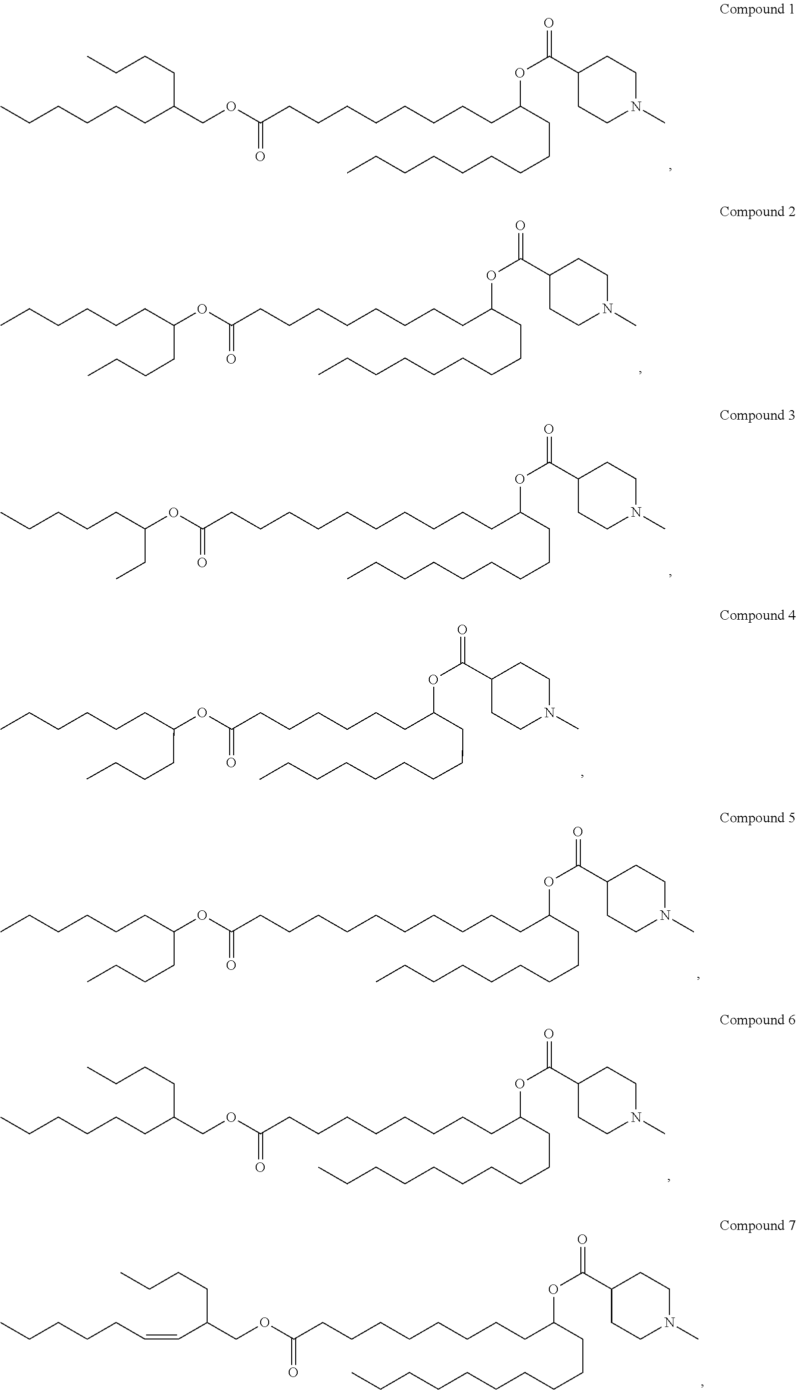 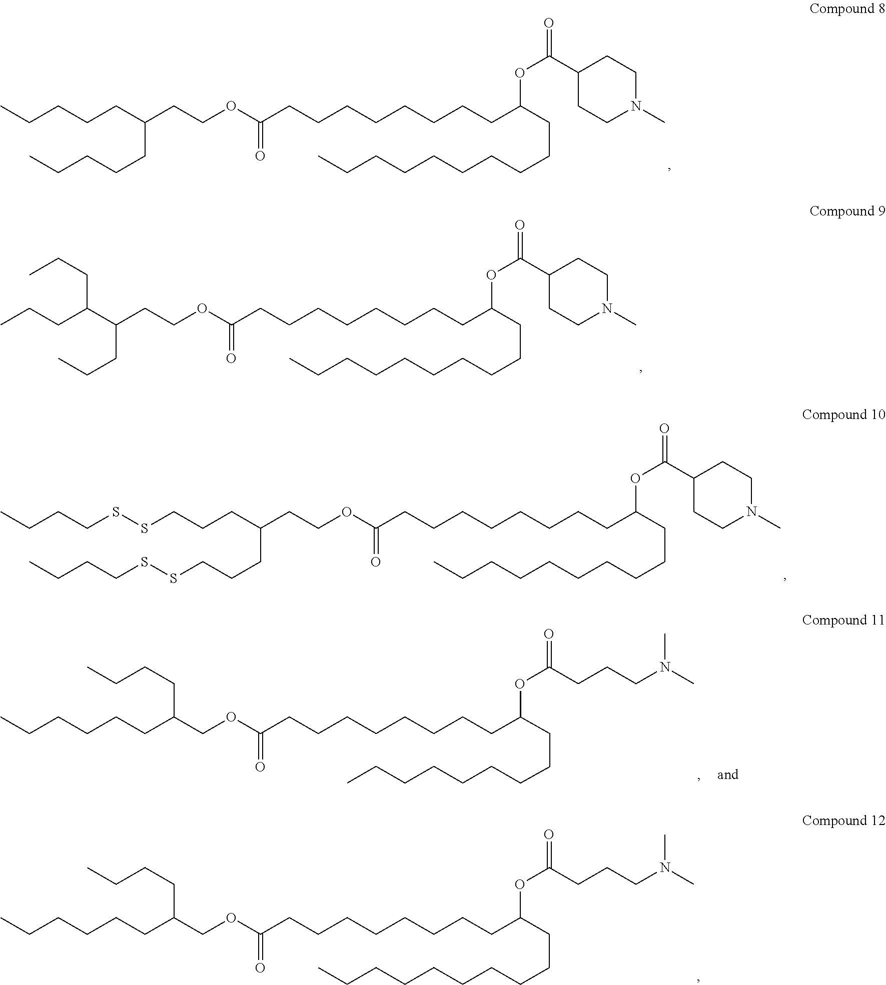
or a pharmaceutically acceptable salt or prodrug thereof.

2. The lipid nanoparticle of claim 1, wherein the lipid component further comprises one or more of a neutral lipid, a structural lipid and a PEGylated lipid.

3. The lipid nanoparticle of claim 2, wherein the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

4. The lipid nanoparticle of claim 2, wherein the structural lipid is selected from the group consisting of cholesterol, fecosterol, sitosterol, campesterol, stigmasterol, brassicasterol, ergosterol, tomatidine, tomatine, ursolic acid and alpha-tocopherol.

5. The lipid nanoparticle of claim 2, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, optionally PEG-c-DOMG, PEG-DMG, PEG-DLPE, PEG-DMPE, PEG-DPPC, and PEG-DSPE.

6. The lipid nanoparticle of claim 1, comprising: about 25 mol % to about 60 mol % of a compound selected from Compounds 1 to 12; about 2 mol % to about 25 mol % neutral lipid; about 18.5 mol % to about 60 mol % structural lipid; and about 0.2 mol % to about 10 mol % of PEGylated lipid.

7. The lipid nanoparticle of claim 1, wherein the messenger RNA is selected from a conventional messenger RNA and a self-amplifying messenger RNA.

8. The lipid nanoparticle of claim 1, wherein the conventional messenger RNA and/or self-amplifying messenger RNA comprise a sequence encoding an antigenic peptide or protein, or a fragment, variant or derivative thereof.

9. The lipid nanoparticle of claim 8, wherein the antigenic peptide or protein is selected from the group consisting of a pathogenic antigen, a tumour antigen, an allergenic antigen or an autoimmune self-antigen.

10. The lipid nanoparticle of claim 9, wherein the pathogenic antigen is derived from a bacterial, viral or protozoological pathogenic organism.

11. The lipid nanoparticle of claim 1, wherein the lipid nanoparticle has a diameter of from about 30 nm to about 160 nm.

12. A lipid nanoparticle composition comprising a plurality of lipid nanoparticles of claim 1, and a pharmaceutically acceptable carrier, excipient or diluent.

13. A method of delivering a messenger RNA to a mammalian cell, including administering the lipid nanoparticle of claim 1 to a subject to thereby contact the cell with the lipid nanoparticle and deliver the messenger RNA to the cell.

14. The method of claim 13, wherein the cell is a cell of a human subject.

15. A method of producing a polypeptide of interest in a mammalian cell, including the step of contacting the cell with a lipid nanoparticle of claim 1, wherein the lipid nanoparticle comprises a conventional messenger RNA or a self-amplifying messenger RNA encoding the polypeptide.

16. A method of treating a disease, disorder or condition in a subject in need of such treatment, comprising administering a lipid nanoparticle of claim 1 to the subject to thereby treat the disease, disorder or condition.

17. The method of claim 16 wherein the disease, disorder or condition is selected from the group consisting of a rare disease, an infectious disease, cancer, a proliferative disease, a genetic disease, an autoimmune disease, diabetes, a neurodegenerative disease, a cardiovascular disease, a reno-vascular disease and a metabolic disease.

18. A vaccine comprising a lipid nanoparticle of claim 1.

19. The vaccine of claim 18, wherein the vaccine is selected from a tumor vaccine, an influenza vaccine, and a SARS vaccine.

20. A vaccine comprising the lipid nanoparticle composition of claim 12.
